SEQUENCE LISTING (16 sequences)

SEQ ID NO: 1 - DNA, 28 bp, Artificial
  FkbDLF primer
tataaagcttcggagccccggtggacct

SEQ ID NO: 2 - DNA, 28 bp, Artificial
  FkbDLR primer
ttaatctagacgtcgcctcgtcgtcgct

SEQ ID NO: 3 - DNA, 28 bp, Artificial
  FkbDRF primer
gtaatctagagtcggctactgcctctac

SEQ ID NO: 4 - DNA, 28 bp, Artificial
  FkbDRR primer
gaatgaattccgacgaacagcggttcct

SEQ ID NO: 5 - DNA, 28 bp, Artificial
  FkbD-MLF primer
tataaagcttcggagccccggtggacct

SEQ ID NO: 6 - DNA, 28 bp, Artificial
  FkbD-MLR primer
ttaatctagacgtcgcctcgtcgtcgct

SEQ ID NO: 7 - DNA, 28 bp, Artificial
  FkbD-MRF primer
tatatctagagacaccgaaggcgcgctc

SEQ ID NO: 8 - DNA, 28 bp, Artificial
  FkbD-MRR primer
ttaagaattcgaacaccgaggccgtcca

SEQ ID NO: 9 - DNA, 28 bp, Artificial
  TcsDLF primer
gctaagcttctcaggcgtctgcggatgc

SEQ ID NO: 10 - DNA, 28 bp, Artificial
  TcsDLR primer
atcggatccttcgctcaccggggctgcc

SEQ ID NO: 11 - DNA, 28 bp, Artificial
  TcsDRF primer
agcagatctggcatgttctggtcagtcc

SEQ ID NO: 12 - DNA, 28 bp, Artificial
  TcsDRR primer
gtcgaattccatgccacgaacgggtcga

SEQ ID NO: 13 - DNA, 22 bp, Artificial
  FkbLLF primer
aataagcttccacgagcccggt

SEQ ID NO: 14 - DNA, 24 bp, Artificial
  FkbLLR primer
aaatctagacacatcgcgttcgac

SEQ ID NO: 15 - DNA, 24 bp, Artificial
  FkbLRF primer
aattctagacacggagaggatctg

SEQ ID NO: 16 - DNA, 21 bp, Artificial
  FkbLRR primer
aaagaattcccaccacccccg

(19) United States
(12) Patent Application Publication
(10) Pub. No.: US 2023/0094227 A1
(43) Pub. Date: Mar. 30, 2023

(54) NOVEL FK506 DERIVATIVE AND COMPOSITION COMPRISING SAME FOR PROMOTING HAIR GROWTH

(71) Applicant: SEOUL NATIONAL UNIVERSITY R&DB FOUNDATION, Seoul (KR)

(72) Inventors: Yeo Joon YOON, Seoul (KR); Myoung-Chong SONG, Seoul (KR); Young Ji YOO, Seoul (KR)

(21) Appl. No.: 17/801,193

(22) PCT Filed: Dec. 23, 2021
(86) PCT No.: PCT/KR2021/019771

§ 371 Date: Aug. 19, 2022

(30) Foreign Application Priority Data
Dec. 24, 2020 (KR) 10-2020-0183249

(51) Int. Cl. A61K 8/49; A61K 8/64; A61Q 7/00; A61Q 5/12; A61Q 5/02

(52) U.S. Cl. CPC A61K 8/492; A61K 8/64; A61Q 7/00; A61Q 5/12; A61Q 5/02

(57) ABSTRACT

The present invention relates to the preparation of 9-deoxo-36,37-dihydro-prolylFK506, 9-deoxo-31-O-demethyl-36,37-dihydro-prolylFK506, 9-deoxo-prolylFK520 or 9-deoxo-31-O-demethyl-prolylFK520, which are novel compounds that can be utilized as the main ingredient of a composition for promoting hair growth, and the use thereof in promoting hair growth.

### TECHNICAL FIELD

The present invention relates to the preparation and utilization of 9-deoxo-36,37-dihydro-prolylFK506, 9-deoxo-31-O-demethyl-36,37-dihydro-prolylFK506, 9-deoxo-prolylFK520 and 9-deoxo-31-O-demethyl-prolylFK520, which are novel compounds that can be used as the main ingredient of a composition for promoting hair growth, and specifically to the preparation method of the novel compounds and a composition for promoting hair growth, including each novel compound as an active ingredient.

### BACKGROUND ART

Hair loss (alopecia) in modern people has become more common not only due to aging or genetic causes, but also due to the influence of acquired factors such as hormone imbalance caused by environmental contamination, smoking, stress from work and changes in dietary patterns. Although the population with hair loss has increased mainly in middle-aged people in their 40s and 50s, the number of young people and females suffering from hair loss has also been increasing in recent years. According to the Korean Society for Alopecia, the population with hair loss accounts for about 10 million in Korea, including potential alopecia patients. The medical expenditure therefor was about 35.5 billion Korean won in 2016, and the number of people with hair loss is increasing abroad as well as in Korea. The market size for hair loss management in the United States, measured by IBIS World Industry Market Research, was $3.6 billion USD in 2016, and is projected to grow at an annual average of 0.7% until 2021. Meanwhile, according to a survey by the Korean Association of Chronic Disease Management, 7 out of 10 adults in Korea recognized hair loss as a disease and thought that hair loss causes direct or indirect losses in social lives. In addition, it was reported that about 23% of adults experience hair loss. Hair loss is classified into non-scarring hair loss, which is temporary hair loss, and scarring hair loss, which occurs when hair follicles or hair roots are permanently destroyed, and non-scarring hair loss is a commonly encountered form of hair loss and is classified into infectious hair loss, traumatic hair loss, inflammatory hair loss, congenital hair loss, endocrine hair loss, neoplastic alopecia, malnutrition hair loss, drug-induced hair loss and hair loss due to structural abnormalities of the hair. In addition, male-pattern hair loss, female-pattern hair loss and alopecia areata also pertain to non-scarring hair loss. As the economy grows and society ages, hair loss has been considered as a disease rather than an uncontrollable phenomenon, and there is a need to develop drugs that are effective in the treatment of hair loss, because it causes psychological anxiety and stress in work and social lives.

However, although the number of people with hair loss is increasing, the exact causes of hair loss have still not been identified, and the situation is that there are no effective methods for preventing hair loss. Due to the current situation, effective techniques for preventing hair loss and promoting hair growth have gained more interest, and many types of hair loss-preventing agents and hair growth agents are currently available on the market. Currently, Minoxidil for topical administration and Propecia for oral administration are drugs that have been approved by the FDA. However, these drugs do not provide permanent therapeutic effects, but only provides effects on delaying the progression of hair loss or maintaining the current hair condition. In addition, it is known that topical hair growth-promoting agents such as Minoxidil may be cumbersome because they need to be constantly applied every day in order to maintain the hair growth-promoting effects, and the hair growth-promoting effects do not appear in many cases compared to the hair growth-promoting agents that are orally administered. Further, Propecia may increase the likelihood of congenital malformations in the fetus when administered to females, and may cause side effects such as sexual dysfunction in men. Therefore, there is a need to develop drugs that are capable of providing fundamental therapeutic effects beyond the level of temporary effects. Moreover, drugs available not only for therapeutic use but also for prophylactic use may be more valuable.

FK506 inhibits the transcription of interleukins by binding to calcineurin (CaN) after binding to FK506-binding protein (FKBP)12 in human cells and inhibiting its activity [Cell 2009, 138, 210], or by binding to FKBP52 (or 51), it exhibits neuroregenerative activity through an unknown mechanism [Nat. Chem. Biol. 2015, 11, 33; Drug Metab. Rev. 1999, 31, 649; U.S. Pat. No. 7,169,564 B1]. In addition, although FK506 has long been known for hair growth activity, its use is limited due to the aforementioned immunosuppressive action [J Invest Dermatol, 1994, 102, 160; Pak J Med Sci, 2009, 25, 833].

### DISCLOSURE

### Technical Problem

An object of the present invention is to provide four novel compounds, isomers thereof or pharmaceutically acceptable salts thereof.

Another object of the present invention is to provide a composition for promoting hair growth, including at least one selected from the four novel compounds as an active ingredient. Herein, the composition for promoting hair growth refers to a composition for ameliorating, preventing or treating hair loss.

Still another object of the present invention is to provide a pharmaceutical composition for promoting hair growth, including at least one selected from the four novel compounds, an isomer thereof or a pharmaceutically acceptable salt thereof as an active ingredient. Herein, the pharmaceutical composition for promoting hair growth refers to a composition for ameliorating, preventing or treating hair loss.

Still another object of the present invention is to provide a biological preparation method of each of the four novel compounds.

Still another object of the present invention is to provide Streptomyces kanamyceticus ΔfkbD,tcsD,fkbL (Accession No. KCTC14171BP) and Streptomyces kanamyceticus ΔfkbD-fkbM,tcsD,fkbL (Accession No. KCTC14170BP), which are production strains that can be used in the biological preparation process of the four novel compounds.

Still another object of the present invention is to provide a quasi-drug composition for preventing or ameliorating hair loss, or promoting hair growth, including at least one selected from the four novel compounds, an isomer thereof or a pharmaceutically acceptable salt thereof as an active ingredient.

Still another object of the present invention is to provide a health functional food composition for preventing or ameliorating hair loss, or promoting hair growth, including at least one selected from the four novel compounds, an isomer thereof or a sitologically acceptable salt thereof as an active ingredient.

Still another object of the present invention is to provide a cosmetic composition for preventing or ameliorating hair loss, or promoting hair growth, including at least one selected from the four novel compounds, an isomer thereof or a cosmetically acceptable salt thereof as an active ingredient.

Still another object of the present invention is to provide the use of at least one selected from the four novel compounds, an isomer thereof or a pharmaceutically acceptable salt thereof in the preparation of a medicament or a quasi-drug for preventing, ameliorating or treating hair loss, or promoting hair growth.

Still another object of the present invention is to provide the use of at least one selected from the four novel compounds, an isomer thereof or a sitologically acceptable salt thereof in the preparation of health functional food for preventing, ameliorating or treating hair loss, or promoting hair growth.

Still another object of the present invention is to provide the use of at least one selected from the four novel compounds, an isomer thereof or a cosmetically acceptable salt thereof in the preparation of a cosmetic for preventing, ameliorating or treating hair loss, or promoting hair growth.

### Technical Solution

Accordingly, the inventors of the present invention have made various efforts to examine the hair growth promoting effects of 9-deoxo-36,37-dihydro-prolylFK506, 9-deoxo-31-O-demethyl-36,37-dihydro-prolylFK506, 9-deoxo-prolylFK520 and 9-deoxo-31-O-demethyl-prolylFK520 (hereinafter, collectively referred to as “four novel compounds”), which are novel compounds that can be utilized as the main ingredient of a pharmaceutical composition for promoting hair growth, to develop a preparation process thereof, to further develop a pharmaceutical composition for preventing hair loss or treating hair growth using these compounds as active ingredients, and completed the present invention by confirming that as a pharmaceutical composition for preventing hair loss or promoting hair growth, this composition has remarkably low immunosuppressive activity compared to the existing FK506 compound or derivatives thereof, and thus can be effectively used without side effects due thereto.

### Advantageous Effects

Since the composition for promoting hair growth, including at least one selected from the four novel compounds according to the present invention as an active ingredient, can provide substantial effects of promoting hair growth in the amelioration, prevention and treatment of hair loss, it can provide more fundamental prophylactic and therapeutic effects.

### DESCRIPTION OF DRAWINGS

FIG. 1 is the results of high performance liquid chromatography analysis for 9-deoxo-36,37-dihydro-prolylFK506.

FIG. 2 is the results of nuclear magnetic resonance analysis (¹H-NMR) for 9-deoxo-36,37-dihydro-prolylFK506.

FIG. 3 is the results of nuclear magnetic resonance analysis (¹³C-NMR) for 9-deoxo-36,37-dihydro-prolylFK506.

FIG. 4 is the results of nuclear magnetic resonance analysis (gCOSY-NMR) for 9-deoxo-36,37-dihydro-prolylFK506.

FIG. 5 is the results of nuclear magnetic resonance analysis (gHSQC-NMR) for 9-deoxo-36,37-dihydro-prolylFK506.

FIG. 6 is the results of nuclear magnetic resonance analysis (gHMBC-NMR) for 9-deoxo-36,37-dihydro-prolylFK506.

FIG. 7 is the results of high performance liquid chromatography analysis for 9-deoxo-31-O-demethyl-36,37-dihydro-prolylFK506.

FIG. 8 is the results of nuclear magnetic resonance analysis (¹H-NMR) for 9-deoxo-31-O-demethyl-36,37-dihydro-prolylFK506.

FIG. 9 is the results of nuclear magnetic resonance analysis (¹³C-NMR) for 9-deoxo-31-O-demethyl-36,37-dihydro-prolylFK506.

FIG. 10 is the results of nuclear magnetic resonance analysis (gCOSY-NMR) for 9-deoxo-31-O-demethyl-36,37-dihydro-prolylFK506.

FIG. 11 is the results of nuclear magnetic resonance analysis (gHSQC-NMR) for 9-deoxo-31-O-demethyl-36,37-dihydro-prolylFK506.

FIG. 12 is the results of nuclear magnetic resonance analysis (gHMBC-NMR) for 9-deoxo-31-O-demethyl-36,37-dihydro-prolylFK506.

FIG. 13 is the results of high performance liquid chromatography analysis for 9-deoxo-prolylFK520.

FIG. 14 is the results of nuclear magnetic resonance analysis (¹H-NMR) for 9-deoxo-prolylFK520.

FIG. 15 is the results of nuclear magnetic resonance analysis (¹³C-NMR) for 9-deoxo-prolylFK520.

FIG. 16 is the results of nuclear magnetic resonance analysis (gCOSY-NMR) for 9-deoxo-prolylFK520.

FIG. 17 is the results of nuclear magnetic resonance analysis (gHSQC-NMR) for 9-deoxo-prolylFK520.

FIG. 18 is the results of nuclear magnetic resonance analysis (gHMBC-NMR) for 9-deoxo-prolylFK520.

FIG. 19 is the results of high performance liquid chromatography analysis for 9-deoxo-31-O-demethyl-prolylFK520.

FIG. 20 is the results of nuclear magnetic resonance analysis (¹H-NMR) for 9-deoxo-31-O-demethyl-prolylFK520.

FIG. 21 is the results of nuclear magnetic resonance analysis (¹³C-NMR) for 9-deoxo-31-O-demethyl-prolylFK520.

FIG. 22 is the results of nuclear magnetic resonance analysis (gCOSY-NMR) for 9-deoxo-31-O-demethyl-prolylFK520.

FIG. 23 is the results of nuclear magnetic resonance analysis (gHSQC-NMR) for 9-deoxo-31-O-demethyl-prolylFK520.

FIG. 24 is the results of nuclear magnetic resonance analysis (gHMBC-NMR) for 9-deoxo-31-O-demethyl-prolylFK520.

FIG. 25 is the results of examining the degree of decrease in immunosuppressive activity of the four novel compounds of the present invention.

FIGS. 26a and 26b show the results of investigation on the hair growth activity of the four novel compounds of the present invention by using human hair follicles, and FIG. 26a confirms the change in hair follicle length, and FIG. 26b confirms the ratio of hair follicles remaining in the anagen stage in the hair cycle.

### BEST MODE

Hereinafter, the present invention will be described in more detail.

Meanwhile, each of the explanations and exemplary embodiments disclosed herein can be applied to other explanations and exemplary embodiments. That is, all combinations of various factors disclosed herein belong to the scope of the present invention. Furthermore, the scope of the present invention should not be limited by the specific disclosure provided hereinbelow.

Additionally, one of ordinary skill in the art will be able to recognize or confirm, based on routine experimentation, many equivalents to the specific embodiments of the present invention described in this application, and such equivalents are intended to be included in the present invention.

In order to solve the above problems, the present invention provides a preparation process of four novel compounds, a composition for preventing, ameliorating or treating hair loss, or promoting hair growth, including each novel compound prepared using the preparation process, and a method for ameliorating, preventing and treating hair loss using the composition.

As an aspect for achieving the above objects, the present invention provides a pharmaceutical composition for preventing or treating hair loss, or promoting hair growth, including any one compound selected from the group consisting of 9-deoxo-36,37-dihydro-prolylFK506 represented by [Chemical Formula 1] below, 9-deoxo-31-O-demethyl-36,37-dihydro-prolylFK506 represented by [Chemical Formula 2] below, 9-deoxo-prolylFK520 represented by [Chemical Formula 3] below and 9-deoxo-31-O-demethyl-prolylFK520 represented by [Chemical Formula 4] below, an isomer thereof or a pharmaceutically acceptable salt thereof as an active ingredient:

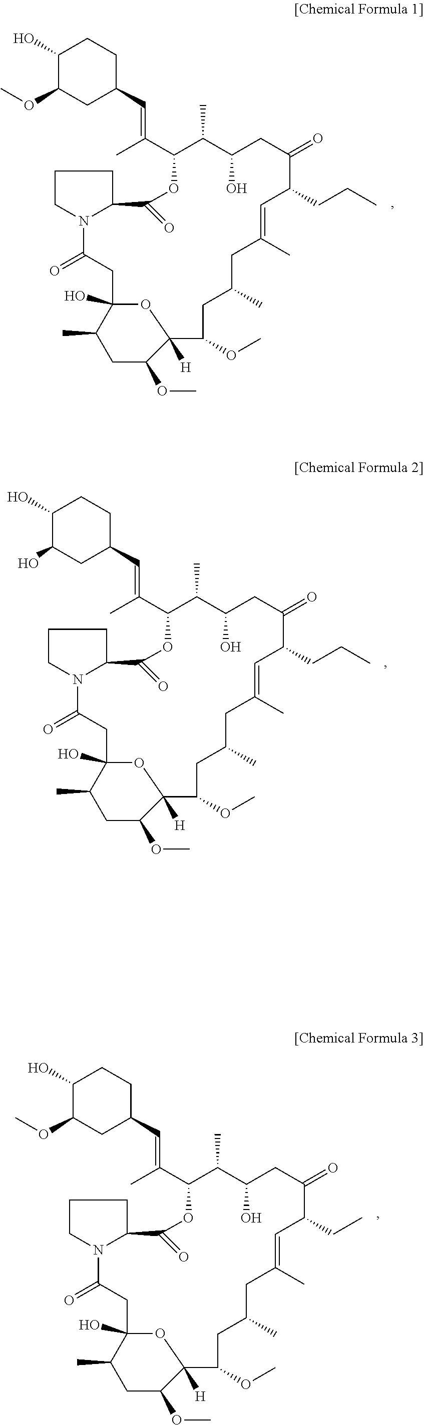

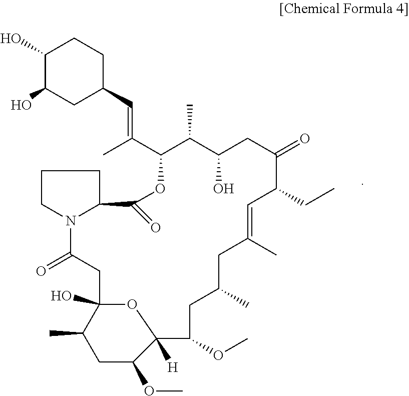 [SMILES: CC[C@@H]1/C=C(\C)C[C@H](C)C[C@H](OC)[C@H]2O[C@@](O)(CC(=O)N3CCC[C@H]3C(=O)O[C@H](/C(C)=C/[C@@H]3CC[C@@H](O)[C@H](O)C3)[C@H](C)[C@@H](O)CC1=O)[C@H](C)C[C@@H]2OC]

As a specific embodiment, the compound of the present invention may include an isomer or a pharmaceutically acceptable salt thereof.

Isomers refer to different compounds with the same chemical formula and may include structural isomers, geometric isomers, optical isomers (enantiomers), stereoisomers and diastereomers.

A pharmaceutically acceptable salt may be any of an organic or inorganic acid addition salt with a concentration relatively nontoxic to a patient, a harmless effective action and a side effect which does not reduce the beneficial effects of a parent compound. For example, the salt may be an acid addition salt formed by a pharmaceutically acceptable free acid. The acid addition salt may be prepared by a conventional method, for example, dissolving a compound in an excess amount of an aqueous acid solution and precipitating the salt using a water-miscible organic solvent such as methanol, ethanol, acetone, or acetonitrile. The salt may be also prepared by heating an aqueous acid or alcohol (e.g., glycol monomethyl ether) and the equimolar compounds and then drying the compound by evaporating or suction filtering the precipitated salt. In this case, an organic or inorganic acid may be used as the free acid. The salt may be a pharmaceutically acceptable metal salt prepared using a base.

As another specific embodiment, the compound of the present invention may be in the form of a solvate or pro-drug which lies within the scope of the present invention. The solvate may preferably include a hydrate or ethanol solvate.

The composition of the present invention may be used as a single formulation, and may be prepared and used as a combined formulation by additionally containing a drug, which is publicly known to have a recognized prophylactic or therapeutic effect on hair loss, in a unit dose form formulated using a pharmaceutically acceptable carrier or excipient, or encapsulated into a multi-dose container.

As used herein, the term “pharmaceutically acceptable carrier” may refer to a carrier or diluent which does not inhibit the biological activities and properties of a compound to be introduced to a biological subject without irritating the biological subject. A type of the carrier which may be used in the present invention is not particularly limited, and any carrier may be used as long as it is a pharmaceutically acceptable carrier commonly used in the art. Non-limiting examples of the carrier include a co-surfactant which may be exemplified by Transcutol, polyethylene glycol, Triacetin and a mixture thereof; Cremophor, Tween, a surfactant which may be exemplified by Myrj, Poloxamer, Pluronic, Lutrol, Imwitor, Span and Labrafil, alone or a mixture thereof; oil which may be exemplified by Miglyol, Captex ethyl oleate alone alone or a mixture thereof; an organic acid which may be exemplified by erythorbic acid and citric acid alone or a mixture thereof and the like. These may be used alone or in mixture of two or more.

In addition, if necessary, other conventional additives such as antioxidants, buffers and/or bacteriostatic agents may be added and used, and diluents, dispersants, surfactants, binders, lubricants and the like may be further added to be used by formulating into dosage forms for injection such as aqueous solutions, suspensions and emulsions, pills, capsules, granules, tablets or the like.

As still another aspect for achieving the above objects, the present invention provides a method for preventing or treating hair loss, including administering the composition to a subject.

As still another aspect for achieving the above objects, the present invention provides a method for promoting hair growth, including administering the composition to a subject.

As used herein, the term “subject” may refer to any animal that has or is likely to have hair loss.

The composition of the present invention may include at least one selected from the four novel compounds, an isomer thereof or a salt thereof, in a pharmaceutically effective amount. As used herein, the term “pharmaceutically effective amount” refers to an amount sufficient to treat a disease at a reasonable benefit/risk ratio applicable to medical treatment, and the compound may be generally administered in an amount of 0.001 to 1,000 mg/kg, preferably, 0.05 to 200 mg/kg, and more preferably, 0.1 to 100 mg/kg in a single or multiple doses per day. For the purpose of the present invention, however, it is preferable that a specific therapeutically effective amount for a particular patient is administered depending upon the type and degree of a desired reaction, whether other formulations are used in some cases, a specific composition, age, body weight, general health condition, sex, diet, the time and route of administration, the secretion rate of a composition, the period of treatment, a drug used either simultaneously or in combination with a specific composition and other various factors and similar factors well-known in the pharmaceutical field.

The administration frequency of the composition of the present invention is not particularly limited thereto, but may be administered once a day or administered several times by dividing the dose.

The composition of the present invention may be administered as an individual therapeutic agent or in combination with other therapeutic agents, and may be administered sequentially or simultaneously with conventional therapeutic agents. In addition, it may be administered a single time or multiple times. In consideration of all of the above factors, it is important to administer an amount that can obtain the maximum effect with a minimum amount while minimizing the occurrence of side effects, and it can be easily determined by those skilled in the art.

As used herein, the term “administration” means introducing the composition of the present invention to a patient by any suitable method, and the administration route of the composition of the present invention may be performed through various routes such as oral or parenteral administration as long as it can reach the target tissue.

The administration method of the composition according to the present invention is not particularly limited, and may follow a method commonly used in the art. As non-limiting examples of the administration method, the composition may be administered by oral administration or parenteral administration. The composition according to the present invention may be prepared in various dosage forms depending on the desired administration mode.

The composition for promoting hair growth of the present invention may be used for the purpose of ameliorating, preventing or treating hair loss.

The hair loss of the present invention includes both non-scarring hair loss, which is temporary hair loss, and scarring hair loss, which appears due to permanent destruction of hair follicles or hair roots, and non-scarring hair loss includes infectious hair loss, traumatic hair loss, inflammatory hair loss, congenital hair loss, endocrine hair loss, neoplastic alopecia, malnutrition hair loss, drug-induced hair loss and hair loss due to structural abnormalities of hair, male-pattern hair loss, female-pattern hair loss and alopecia areata.

As used herein, the term “amelioration” may refer to any actions of delaying the progression of hair loss or alleviating the symptoms of hair loss by administering the composition according to the present invention to a subject.

As used herein, the term “prevention” may refer to any actions of suppressing or delaying the onset of hair loss by administering the composition according to the present invention to a subject.

As used herein, the term “treatment” may refer to any actions of improving or alleviating the symptoms of hair loss by administering the composition of the present invention to a subject suspected of developing hair loss.

As still another aspect for achieving the above objects, the present invention provides a biological preparation process of four novel compounds, 9-deoxo-36,37-dihydro-prolylFK506, 9-deoxo-31-O-demethyl-36,37-dihydro-prolylFK506, 9-deoxo-prolylFK520 and 9-deoxo-31-O-demethyl-prolylFK520.

In the biological preparation process, in general, the culture temperature adopted in the culturing process of the genus Streptomyces is used. As a suitable culture temperature for the implementation of the present invention, 23 to 30° C. may be preferably applied, and more preferably, a culture temperature of 25 to 28° C. may be applied.

Further, in the preparation process, the pH of the culture process is maintained between 6.5 to 9, and preferably, the culture pH is maintained at 7 to 8.

Meanwhile, in the preparation process, it is important to maintain a high level of dissolved oxygen in the culture medium, and when the dissolved oxygen level at the beginning of the culture is 100%, it is important to maintain the dissolved oxygen level at 30% or more until the end of the culture. In order to implement this, it is generally preferable to stir at a level of 800 to 1,500 rpm.

The extraction of the four novel compounds produced from the cultured cell body in the preparation process is achieved through the implementation of a primary extraction process, a secondary extraction process and a tertiary extraction process, and in the present invention, the organic solvent extraction method is used as the primary extraction process. In this case, examples of the solvent that can be used include ethyl acetate, methanol, acetone and the like, but the use of ethyl acetate or methanol is preferable. In addition, silica gel chromatography is used as the secondary extraction process, and in this case, examples of the solvent that can be used preferably include methanol, methylene chloride, n-hexane or ethyl acetate. In addition, chromatography is used as the tertiary extraction process, and in this case, examples of the solvent that can be used may include acetonitrile, ammonium acetate buffer, acetic acid, formic acid and the like, but the use of acetonitrile is preferable. The application of this method facilitates the recovery of the four novel compounds and also increases the yield.

As still another aspect for achieving the above objects, the present invention provides Streptomyces kanamyceticus ΔfkbD,tcsD,fkbL (Accession No. KCTC14171BP) and Streptomyces kanamyceticus ΔfkbD-fkbM,tcsD,fkbL (Accession No. KCTC14170BP), which are production strains that can be used for the preparation of the four novel compounds.

Still another object of the present invention is to provide a quasi-drug composition for preventing or ameliorating hair loss, or promoting hair growth, including at least one selected from the four novel compounds, an isomer thereof or a pharmaceutically acceptable salt thereof as an active ingredient.

When the composition including at least one selected from the four novel compounds is added to a quasi-drug composition for the purpose of promoting hair growth, the compound may be added as it is or may be used together with other quasi-drug ingredients, and may be appropriately used according to a conventional method. The mixing amount of the active ingredient may be appropriately determined according to the purpose of use.

As used herein, the term “quasi-drug composition” refers to fibers, rubber products or similar articles used for the purpose of treating, alleviating, handling or preventing diseases in humans or animals; non-appliance, non-machinery or similar articles which have insignificant influences on or do not directly act upon human bodies; preparations used for the purpose of disinfection or pest control, and similar purpose thereto for the prevention of infectious diseases, and the quasi-drug composition refers to articles used for the purposes of diagnosis, treatment, alleviation, handling or prevention of diseases of human beings or animals, excluding appliances, machinery or equipment; or articles, other than appliances, machinery or equipment, used for the purpose of exerting pharmacological effects upon the structure or functions of human beings or animals. Specifically, the quasi-drug composition may be a skin external agent or a personal hygiene product.

Although not particularly limited thereto, the skin external agent may be prepared specifically as an ointment, a lotion, a spray, a patch, a cream, a powder, a suspension, a gel agent or a form of gel. The personal hygiene product may be, but is not particularly limited to, specifically a soap, a cosmetic, a wet tissue, a tissue, a shampoo, a skin cream, a face cream, a toothpaste, a lipstick, a perfume, a makeup base, a foundation, a blusher, a mascara, an eye shadow, a sunscreen lotion, a hair care product, an air freshener gel or a wash gel.

In addition, as another example of the quasi-drug composition of the present invention, a disinfectant cleaner, a shower foam, an ointment, a wet tissue, a coating agent and the like may be exemplified, but the present invention is not limited thereto, and it may be appropriately selected from conventional techniques known in the art.

Preferred types of the formulated quasi-drug composition of the present invention include a scalp tonic, a scalp lotion, a scalp cream, a scalp serum, a scalp essence, a scalp ampoule, a scalp treatment, a scalp conditioner, a scalp shampoo, a scalp pack, a hair tonic, a hair lotion, a hair cream, a hairspray, a hair mousse, a hair gel, a hair conditioner, a hair shampoo, a hair conditioner, a hair pack, a hair treatment, an eyebrow hair growth agent, an eyelash hair growth agent, an eyelash nutritional supplement, a pet shampoo or a pet rinse, but the present invention is not limited thereto.

The quasi-drug composition of the present invention may further include a pharmaceutically acceptable carrier, excipient or diluent if necessary in addition to the above components. The pharmaceutically acceptable carrier, excipient or diluent is not limited as long as it does not impair the effects of the present invention, and may include, for example, fillers, extenders, binders, wetting agents, disintegrants, surfactants, lubricants, sweeteners, fragrances, preservatives and the like.

In an exemplary embodiment of the present invention, the hair growth promoting effect of the four novel compounds was confirmed, and it was confirmed that it can be used as a quasi-drug composition for preventing hair loss and promoting hair growth.

Still another object of the present invention is to provide a health functional food composition for preventing or ameliorating hair loss, or promoting hair growth, including at least one selected from the four novel compounds, an isomer thereof or a sitologically acceptable salt thereof as an active ingredient.

The composition including at least one compound selected from the four novel compounds may be prepared and consumed in the form of food that can prevent or alleviate hair loss-related diseases by confirming the hair growth promoting effect.

As used herein, the term “health functional food or nutraceutical food” is the same as food for special health use, functional food and health food, and refers to food having high medicinal and medical effects, which is processed to effectively exert a body-regulating function as well as to supply nutrients. The food may be prepared in various forms such as tablets, capsules, powders, granules, liquids and pills to obtain a beneficial effect in preventing or alleviating diseases related to hair loss.

The health functional food of the present invention may be prepared by way of a method commonly used in the art and by adding raw materials and ingredients which are generally added in the art during the preparation. In addition, the formulation of the health functional food may be prepared without limitation as long as the formulation is acceptable as health functional food. The health functional food of the present invention may be prepared in various types of formulations, and unlike general drugs, the health food composition includes food as a raw material, and therefore, it has advantages of being free from side effects that may occur when taken for a long period of time. In addition, since the health functional food composition is excellent in portability, the health functional food of the present invention may be taken as a supplement agent for enhancing hair growth-promoting effects.

Specifically, the food is food prepared by adding at least one compound selected from the four novel compounds or an isomer thereof to a food material such as beverages, teas, flavors, gums, confectionery or the like, or food prepared as a capsule, powder or suspension, which may be used, for example, as various foods such as beverages, gum, teas, vitamin complexes and health functional food.

The food may be prepared in formulations such as tablets, granules, powders, capsules, liquid solutions and pills according to any known manufacturing methods, and the amount of the composition of the present invention may be adjusted according to the formulation. The other ingredients except for the at least one compound selected from the four novel compounds according to the present invention, as an active ingredient, are not particularly limited, and the composition may further include various flavoring agents or natural carbohydrates as an additional ingredient.

Examples of the natural carbohydrate include typical sugars, for example, monosaccharides such as glucose and fructose; disaccharides such as maltose and sucrose; and polysaccharides such as dextrin and cyclodextrin, and sugar alcohols such as xylitol, sorbitol, erythritol and the like. In addition to the foregoing, flavoring agents including natural flavoring agents such as taumatin and stevia extracts (e.g., rebaudioside A and glycyrrhizin) and synthetic flavoring agents (e.g., saccharin and aspartame) may advantageously be used.

Additionally, the composition or the health functional food of the present invention may include a variety of nutrients, vitamins, minerals (electrolytes), synthetic and natural flavoring agents, colorants and fillers (cheese, chocolate, etc.), pectic acid or salts thereof, alginic acid or salts thereof, organic acids, protective colloidal thickeners, pH modifiers, stabilizers, preservatives, glycerin, alcohols and carbonating agents used in carbonated beverages.

In addition, the health functional food of the present invention may include fruit pulp for natural fruit juice, fruit juice drinks and vegetable drinks. These ingredients may be used alone or in combination.

When the formulation is powder, the carrier may be lactose, talc, silica, aluminum hydroxide, calcium silicate, polyamide powder or any mixture thereof.

When the formulation of the present invention is a solution or emulsion, the carrier may be a solvent, a solubilizer or an emulsifier, for example, water, ethanol, isopropanol, ethyl carbonate, ethyl acetate, benzyl alcohol, benzyl benzoate, propylene glycol, 1,3-butyl glycol oil, specifically cotton seed oil, peanut oil, corn seed oil, olive oil, castor oil, sesame oil, glycerol, an aliphatic ester, polyethylene glycol or a fatty acid ester of sorbitan.

When the formulation is a suspension, the carrier may be a liquid diluent such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar or tragacanth.

Still another object of the present invention is to provide a cosmetic composition for preventing or ameliorating hair loss, or promoting hair growth, including at least one selected from the four novel compounds, an isomer thereof or a cosmetically acceptable salt thereof as an active ingredient.

As used herein, the “cosmetic composition” may be prepared in any formulation that is commercially manufactured, for example, a solution, an emulsion, a suspension, a paste, a cream, a lotion, a gel, a powder, a spray, a surfactant-containing cleaner, an oil, a soap, a liquid cleaner, a bath bomb, a foundation, a makeup base, an essence, a nourishing lotion, a foam, a pack, a softening water, a sunscreen cream, a sun oil or the like.

When the formulation of the present invention is a solution or emulsion, the carrier may be a solvent, a solubilizer or an emulsifier such as water, ethanol, isopropanol, ethyl carbonate, ethyl acetate, benzyl alcohol, benzyl benzoate, propylene glycol, 1,3-butyl glycol oil, glycerol, an aliphatic ester, polyethylene glycol or a fatty acid ester of sorbitan.

When the formulation is a suspension, the carrier may be a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth or the like.

When the formulation of the present invention is a paste, a cream or a gel, the carrier may be animal oil, vegetable oil, wax, paraffin, starch, tragacanth, a cellulose derivative, polyethylene glycol, silicone, bentonite, silica, talc, zinc oxide or the like.

When the formulation of the present invention is a powder or spray, the carrier may be lactose, talc, silica, aluminum hydroxide, calcium silicate or polyamide powder. Particularly, in the case of a spray, the formulation may further include a propellant such as a chlorofluorohydrocarbon, propane/butane or dimethyl ether.

When the formulation of the present invention is a surfactant-containing cleaner, the carrier may be an aliphatic alcohol sulfate, aliphatic alcohol ether sulfate, sulfosuccinic acid monoester, isethionate, imidazolinium derivative, methyl taurate, sarcosinate, fatty acid amide ether sulfate, alkyl amido betaine, aliphatic alcohol, fatty acid glyceride, fatty acid diethanolamide, vegetable oil, lanolin derivative, ethoxylated glycerol fatty acid ester or the like.

In an embodiment of the present invention, since the hair growth promoting effect of the four novel compounds was confirmed, it was confirmed that the composition including the same can be used as a cosmetic composition for preventing hair loss and promoting hair growth.

Furthermore, the present invention provides the use of at least one selected from the four novel compounds, an isomer thereof or a pharmaceutically acceptable salt thereof in the preparation of a composition for preventing, ameliorating or treating hair loss, or promoting hair growth.

In the use of the present invention, the composition may be in the form of a medicament, a quasi-drug, health functional food and/or a cosmetic, and the salt may be in the form of a pharmaceutically acceptable salt, a sitologically acceptable salt or a cosmetically acceptable salt.

Hereinafter, the present invention will be described in more detail with reference to the following examples. However, these examples are only for illustrating the present invention, and the scope of the present invention is not limited thereto.

### MODES OF THE INVENTION

### EXAMPLE 1

### Preparation of 9-deoxo-36,37-dihydro-prolylFK506

The in-frame deletion method by double cross-over homologous recombination according to the method described in Ban, Y. H. et al. (J. Nat. Prod. 2013, 76, 1091-1098) was used for Streptomyces kanamyceticus, which is a strain producing FK506, to cause the inactivation of fkbD, tcsD and fkbL genes to construct Streptomyces kanamyceticus ΔfkbD,tcsD,fkbL (Accession No. KCTC14171BP), which is a production strain of 9-deoxo-36,37-dihydro-prolylFK506.

Specifically describing, in order to construct a deletion mutant of the fkbD, tcsD and fkbL genes in the Streptomyces kanamyceticus strain producing FK506, each gene was cloned into the pKC1139 vector and transferred to Escherichia coli ET12567/pUZ8002, and it was transformed into the FK506 producing strain Streptomyces kanamyceticus through conjugation.

The strain production method can be more specifically described as the construction of in-frame gene deletion plasmids and the construction of gene deletion strains.

For the construction of in-frame gene deletion plasmids, E. coli-Streptomyces shuttle vector pKC1139 was used for in-frame gene deletion. Plasmid construction was performed by PCR amplification of the left- and right-flanking fragments of the target gene for deletion from Fosmid DNA derived from Streptomyces kanamyceticus. For deletion of the fkbD gene, a primer pair FkbDLF/FkbDLR for the left-adjacent fragment and a primer pair FkbDRF/FkbDRR for the right-adjacent fragment were designed. For deletion of the tcsD gene, a primer pair TcsDLF/TcsDLR for the left-adjacent fragment and a primer pair TcsDRF/TcsDRR for the right-adjacent fragment were designed. For deletion of the fkbL gene, a primer pair FkbLLF/FkbLLR for the left-adjacent fragment and a primer pair FkbLRF/FkbLRR for the right-adjacent fragment were designed. All PCR fragments were isolated, digested with HindIII-XbaI or XbaI-EcoRI, and then cloned into pKC1139 vector. Information on the strains, plasmids and primers used in this example is presented in Tables 1 and 2 below.

The plasmids used to construct the gene deletion strains are summarized in Table 1. The plasmid for removing C9 hydroxylase, pΔfkbD, was transferred to Escherichia coli ET12567/pUZ8002 and then introduced into Streptomyces kanamyceticus by conjugation to delete the target gene by homologous recombination. Strains in which a single crossover occurred between the deletion plasmid and the Streptomyces kanamyceticus chromosome were selected by culturing an apramycin-resistant transconjugant in the presence of apramycin at 37° C. (non-growth tolerance temperature for pSG5-based replicon). Afterwards, the obtained colonies were propagated three times without selection at 28° C. to allow a second crossover. Two achieved double crossover mutation, that is, ΔfkbD, was selected as an apramycin-sensitive expression trait, which was then confirmed by PCR and optionally by Southern block analysis.

A plasmid for modifying the C21 side chain, pΔtcsD, was introduced into the constructed Streptomyces kanamyceticus ΔfkbD lacking the fkbD gene, and the tcsD gene was deleted using the same method as the fkbD gene deletion method. ΔfkbD,tcsD was selected as an apramycin-sensitive expression trait and then confirmed by PCR and optionally by Southern block analysis. By introducing pΔfkbL, which is a plasmid for forming a C1 prolyl ring, into the additionally constructed Streptomyces kanamyceticus ΔfkbD,tcsD, in which the fkbD and tcsD genes were deleted, the fkbL gene was deleted using the same method as the fkbD and tcsD gene deletion method. ΔfkbD,tcsD,fkbL was selected as an apramycin-sensitive expression trait and then confirmed by PCR.

The constructed fkbD, tcsD and fkbL gene deletion strain, Streptomyces kanamyceticus ΔfkbD,tcsD,fkbL was deposited at the Korean Collection for Type Cultures (KCTC) on Apr. 14, 2020 (Accession No. KCTC14171BP).

TABLE 1
Information on strains and plasmids used

| Strain/vector | Relevant characteristic |
|---|---|
| Bacterial strains | |
| Escherichia coli | |
| DH5α | Host for general cloning |
| ET12567/pUZ8002 | Donor strain for intergeneric conjugation |
| | between E. coli and Streptomyces |
| Streptomyces | |
| Streptomyces | Wild-type FK506 producing strain |
| kanamyceticus | |
| ΔfkbD, tcsD, fkbL | Mutant of Streptomyces kanamyceticus with an |
| | in-frame deletion of fkbD, tcsD, fkbL |
| ΔfkbD-fkbM, tcsD, | Mutant of Streptomyces kanamyceticus with an |
| fkbL | in-frame deletion of fkbD-fkbM, tcsD, fkbL |
| Plasmid | |
| pKC1139 | High-copy-number temperature-sensitive E. coli- |
| | Streptomyces shuttle vector |
| pΔfkbD | Deletion plasmid with in-frame deletion of 51- |
| | bp internal fkbD fragment |
| pΔfkbD-fkbM | Deletion plasmid with in-frame deletion of 1100- |
| | bp internal fkbDM fragment |
| pΔtcsD | Deletion plasmid with in-frame deletion of 1154- |
| | bp internal tcsD fragment |
| pΔfkbL | Deletion plasmid with in-frame deletion of 873- |
| | bp internal fkbL fragment |
| Strain/vector | Relevant characteristic |

TABLE 2
Information on primers used

| | Sequence 5′ to 3′ | SEQ | Restric- |
|---|---|---|---|
| | (Restriction | ID | tion |
| Primer | site underlined) | NO | enzyme |
| FkbDLF | TATAAAGCTTCGGAGCCCCGGTGGACCT | 1 | HindIII |
| FkbDLR | TTAATCTAGACGTCGCCTCGTCGTCGCT | 2 | XbaI |
| FkbDRF | GTAATCTAGAGTCGGCTACTGCCTCTAC | 3 | XbaI |
| FkbDRR | GAATGAATTCCGACGAACAGCGGTTCCT | 4 | EcoRI |
| FkbD-MLF | TATAAAGCTTCGGAGCCCCGGTGGACCT | 5 | HindIII |
| FkbD-MLR | TTAATCTAGACGTCGCCTCGTCGTCGCT | 6 | XbaI |
| FkbD-MRF | TATATCTAGAGACACCGAAGGCGCGCTC | 7 | XbaI |
| FkbD-MRR | TTAAGAATTCGAACACCGAGGCCGTCCA | 8 | EcoRI |
| TcsDLF | GCTAAGCTTCTCAGGCGTCTGCGGATGC | 9 | HindIII |
| TcsDLR | ATCGGATCCTTCGCTCACCGGGGCTGCC | 10 | BamHI |
| TcsDRF | AGCAGATCTGGCATGTTCTGGTCAGTCC | 11 | BglI |
| TcsDRR | GTCGAATTCCATGCCACGAACGGGTCGA | 12 | EcoRI |
| FkbLLF | AATAAGCTTCCACGAGCCCGGT | 13 | HindIII |
| FkbLLR | AAATCTAGACACATCGCGTTCGAC | 14 | XbaI |
| FkbLRF | AATTCTAGACACGGAGAGGATCTG | 15 | XbaI |
| FkbLRR | AAAGAATTCCCACCACCCCCG | 16 | EcoRI |

9-Deoxo-36,37-dihydro-prolylFK506 was prepared through the culture of the constructed production strain Streptomyces kanamyceticus ΔfkbD,tcsD,fkbL (Accession No. KCTC14171BP). It is specifically described as follows. In a 250 mL baffled flask, 50 mL of R2YE medium (sucrose 103 g/L, glucose 10 g/L, potassium sulfate 0.25 g/L, magnesium chloride hexahydrate 10.12 g/L, casamino acid 0.1 g/L, yeast extract (10%) 50 mL/L, TES buffer (5.73%, pH 7.2) 100 mL/L, potassium phosphate (0.5%) 10 mL/L, calcium chloride dihydrate (3.68%) 80 mL/L, L-proline (20%) 15 mL/L, trace element solution 2 mL/L, sodium hydroxide (1 N) 5 mL/L) was added, and the production strain was inoculated thereto, and pre-culture was carried out for two days in a rotary shaking incubator at 28° C. and 180 rpm. Next, 10 mL of the culture medium, which was pre-cultured for two days, was inoculated into a 3 L Erlenmeyer flask to which 1 L of R2YE medium was added. After inoculation, culture was performed for 6 days at 28° C. and 180 rpm. After culturing for 6 days, 9-deoxo-36,37-dihydro-prolylFK506, which was produced through the primary recovery process, was extracted.

The primary recovery process was carried out as follows. First, the same amount of methanol was added to the culture medium and mixed for 30 minutes and centrifuged to remove cells, and the extract from which the cells were removed was concentrated using a rotary evaporator. Then, the concentrated extract was dissolved in water, ethyl acetate was added in a double volume, mixed well and then left to stand until the layers were separated. After the layers were separated, the organic solvent layer of the upper layer was recovered and concentrated using a rotary evaporator, and the weight after concentration was measured. The extract obtained by performing the primary recovery process was passed through a column filled with silica gel. In this case, the amount of silica gel was 15 times the weight of the extract in the primary recovery process, and the mobile phase was used at 5 ratios of n-hexene and ethyl acetate (fraction 1. 1:1, fraction 2. 1:2, fraction 3. 1:3, fraction 4. 0:1, fraction 5. methanol). In fraction 3, 9-deoxo-36,37-dihydro-prolylFK506 was identified. Fraction 3 thus obtained was concentrated using a rotary evaporator and finally purified using HPLC.

It was freeze-dried to obtain 9-deoxo-36,37-dihydro-prolylFK506, which is a substance represented by [Chemical Formula 1], in powder form.

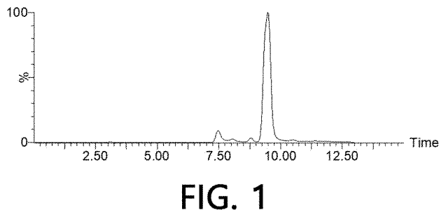

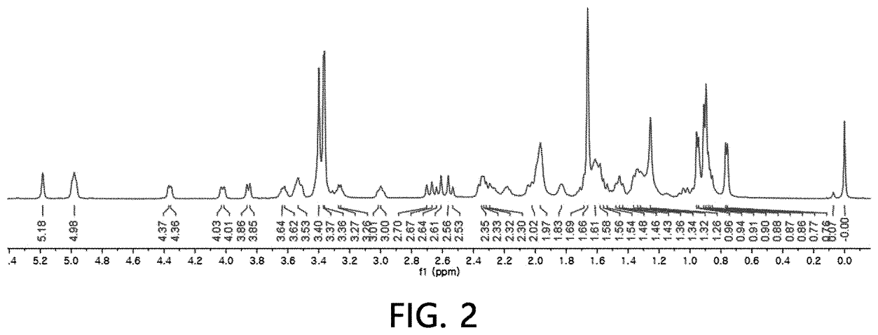

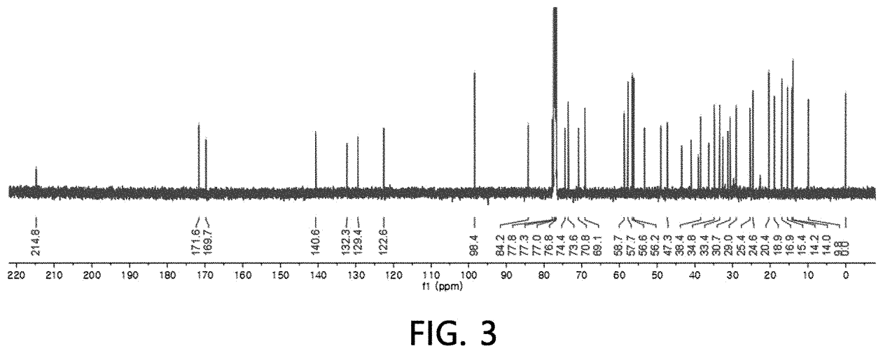

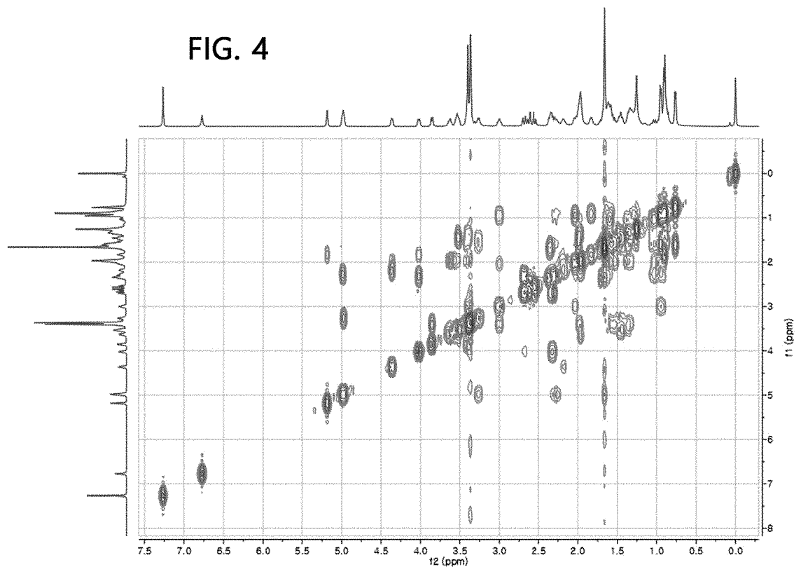

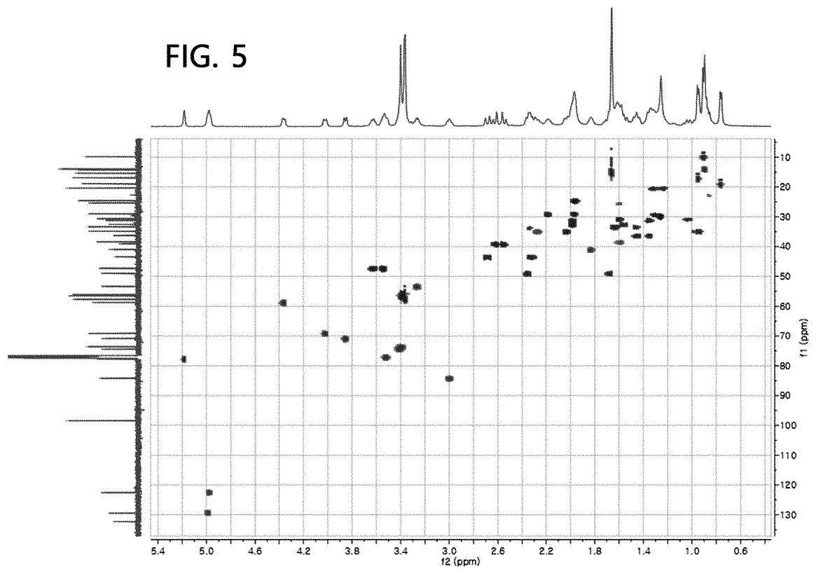

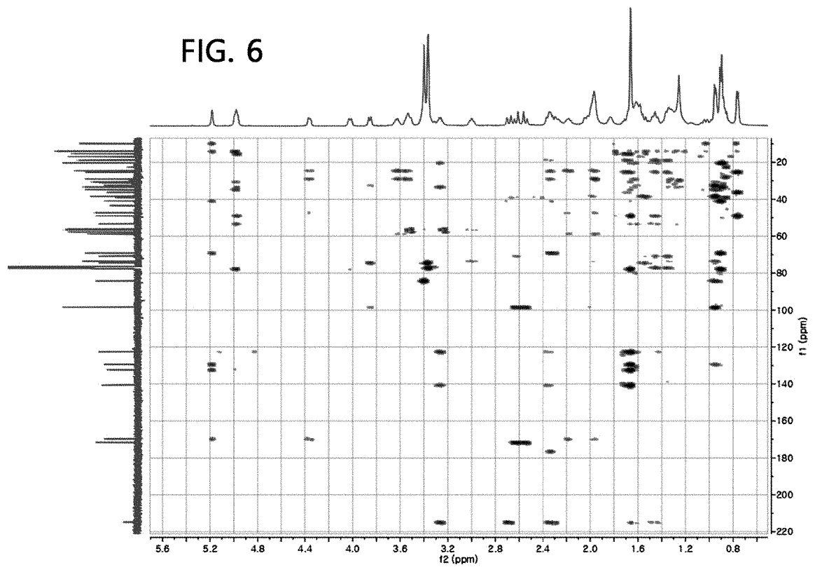

The confirmation of the prepared 9-deoxo-36,37-dihydro-prolylFK506 was carried out as follows. Specifically, high performance liquid chromatography analysis, mass spectrometry and nuclear magnetic resonance analysis were performed. The analysis results for 9-deoxo-36,37-dihydro-prolylFK506 are summarized in Table 3 and FIGS. 1 to 6, and from these results, it was confirmed that 9-deoxo-36,37-dihydro-prolylFK506 was produced from the production strain Streptomyces kanamyceticus ΔfkbD,tcsD,fkbL.

The analysis results for 9-deoxo-36,37-dihydro-prolylFK506 (molecular formula: C₄₃H₇₁NO₁₁, molecular weight: 777.50) are shown in Table 3 below.

TABLE 3

| | Analysis results | | |
|---|---|---|---|
| | (ESI-HR-MS) Calcd. for C₄₃H₇₁NNaO₁₁⁺: | | |
| Analysis method | 800.4919, found: m/z 800.4924 | | |
| Mass spectrometry | No. | carbon (ppm) | proton (ppm) |
| Nuclear magnetic | 1 | 169.7 | |
| resonance analysis | 2 | 58.7 | 4.36 (1H, brd, J = 5.0 Hz) |
| | 3 | 29.0 | 1.97 (1H, m), 2.19 (1H, m) |
| | 4 | 24.6 | 1.97 (1H, m), 1.98 (1H, m) |
| | 5 | 47.3 | 3.53 (1H, m), 3.63 (1H, m) |
| | 6 | | |
| | 7 | | |
| | 8 | 171.6 | |
| | 9 | 39.0 | 2.55 (1H, d, J = 15.0 Hz), |
| | | | 2.62 (1H, d, J = 15.0 Hz) |
| | 10 | 98.4 | |
| | 11 | 38.4 | 1.59 (1H, m) |
| | 12 | 32.5 | 1.56 (1H, m), 1.98 (1H, m) |
| | 13 | 74.4 | 3.40 (1H, m) |
| | 14 | 70.8 | 3.85 (1H, brd, J = 10.0 Hz) |
| | 15 | 77.4 | 3.53 (1H, m) |
| | 16 | 36.3 | 1.34 (1H, m), 1.45 (1H, m) |
| | 17 | 25.4 | 1.60 (1H, m) |
| | 18 | 49.0 | 1.67 (1H, m), 2.36 (1H, m) |
| | 19 | 140.6 | |
| | 20 | 122.6 | 4.98 (1H, d, J = 5.0 Hz) |
| | 21 | 53.4 | 3.26 (1H, m) |
| | 22 | 214.8 | |
| | 23 | 43.4 | 2.31 (1H, brd J = 15.0 Hz), |
| | | | 2.68 (1H, brd J = 15.0 Hz) |
| | 24 | 69.1 | 4.02 (1H, m) |
| | 25 | 40.9 | 1.82 (1H, m) |
| | 26 | 77.8 | 5.18 (1H, brs) |
| | 27 | 132.3 | |
| | 28 | 129.4 | 4.97 (1H, d, J = 5.0 Hz) |
| | 29 | 34.8 | 2.26 (1H, m) |
| | 30 | 34.8 | 0.94 (1H, m), 2.04 (1H, m) |
| | 31 | 84.2 | 3.00 (1H, m) |
| | 32 | 73.6 | 3.40 (1H, m) |
| | 33 | 31.2 | 1.33 (1H, m), 1.98 (1H, m) |
| | 34 | 30.7 | 1.03 (1H, m), 1.59 (1H, m) |
| | 35 | 33.4 | 1.46 (1H, m), 1.63 (1H, m) |
| | 36 | 20.4 | 1.22 (2H, m) |
| | 37 | 14.0 | 0.88 (3H, t, J = 7.5 Hz) |
| | 38 | 16.9 | 0.95 (3H, d, J = 6.5 Hz) |
| | 39 | 18.9 | 0.76 (3H, d, J = 6.5 Hz) |
| | 40 | 15.4 | 1.63 (3H, s) |
| | 41 | 9.8 | 0.85 (3H, d, J = 6.5 Hz) |
| | 42 | 14.2 | 1.65 (3H, s) |
| | 43 | 56.2 | 3.36 (3H, s) |
| | 44 | 57.7 | 3.37 (3H, s) |
| | 45 | 56.6 | 3.40 (3H, s) |

From ¹H and ¹³C-NMR, one ketone carbon (δ_{C} 214.8), two carbonyl carbons (δ_{C} 171.6, 169.7) and two olefine skeletons (δ_{C} 140.6, 122.6; δ_{C} 132.3, 129.4) were identified as characteristic functional groups, and dioxygenated quaternary carbon (δ_{C} 98.4), seven oxygenated methine carbons (δ_{C} 84.2, 77.8, 77.4, 74.4, 73.6, 70.8, 69.1), and three methoxy carbons (δ_{C} 57.7, 56.6, 56.2) were observed. In addition, six methyl carbons (δ_{C} 18.9, 16.9, 15.4, 14.2, 14.0, 9.8) were observed, and all of 43 carbons were observed in the FK506 derivative.

In order to determine the exact structure, 2D-NMR was confirmed. As a result of determining the connection of protons from gCOSY, it was confirmed from the coupling between H-2 to H-4 that this compound had a prolyl skeleton without a CH₂ functional group, not FK506 of the pipecolyl skeleton. From the gHMBC data, it was confirmed that this compound was a backbone reduced to CH₂ instead of ketone at C-9 from the correlation of H-9 (δ_{H} 2.55, 2.62) with C-8 (δ_{C} 171.6) and C-10 (δ_{C} 98.4). Since exomethylene between C36 to C37, which are the basic framework of FK506, was not observed and H37, which was observed as a triplet in gCOSY 2D-NMR, showed a coupling correlation between H-36a/b and H-36a/b and H35a/b, respectively, it was confirmed that the skeleton of C-36 to C-37 was dehydrogenated.

In summary, it was confirmed that the three methoxy functional groups had a structure in which methoxy was present at C-13, C-15 and C-31. Taken together, it was confirmed that this compound was 9-deoxo-36,37-dihydro-prolylFK506.

### EXAMPLE 2

### Preparation of 9-deoxo-31-O-demethyl-36,37-dihydro-prolylFK506

The in-frame deletion method by double cross-over homologous recombination according to the method described in Ban, Y. H. et al. (J. Nat. Prod. 2013, 76, 1091-1098) was used for Streptomyces kanamyceticus, which is a strain producing FK506, to cause the inactivation of fkbD-fkbM, tcsD and fkbL genes to construct Streptomyces kanamyceticus ΔfkbD-fkbM,tcsD,fkbL (Accession No. KCTC14170BP), which is a production strain of 9-deoxo-31-O-demethyl-36,37-dihydro-prolylFK506.

Specifically describing, in order to construct a deletion mutant of the fkbD-fkbM,tcsD and fkbL genes in the Streptomyces kanamyceticus strain producing FK506, each gene was cloned into the pKC1139 vector and transferred to Escherichia coli ET12567/pUZ8002, and then, it was transformed into the FK506 producing strain Streptomyces kanamyceticus through conjugation.

The strain production method can be more specifically described as the construction of in-frame gene deletion plasmids and the production of gene deletion strains.

For the construction of in-frame gene deletion plasmids, E. coli-Streptomyces shuttle vector pKC1139 was used for in-frame gene deletion. Plasmid construction was performed by PCR amplification of the left- and right-flanking fragments of the target gene for deletion from Fosmid DNA derived from Streptomyces kanamyceticus. For deletion of the fkbD-fkbM gene, a primer pair FkbD-MLF/FkbD-MLR for the left-adjacent fragment and a primer pair FkbD-MRF/FkbD-MRR for the right-adjacent fragment were designed. For deletion of the tcsD gene, a primer pair TcsDLF/TcsDLR for the left-adjacent fragment and a primer pair TcsDRF/TcsDRR for the right-adjacent fragment were designed. For deletion of the fkbL gene, a primer pair FkbLLF/FkbLLR for the left-adjacent fragment and a primer pair FkbLRF/FkbLRR for the right-adjacent fragment were designed. All PCR fragments were isolated, digested with HindIII-XbaI or XbaI-EcoRI and then cloned into pKC1139 vector. Information on the strains, plasmids and primers used in this example is presented in Tables 1 and 2 above.

The plasmids used to construct the gene deletion strain are summarized in Table 1. The plasmid, pΔfkbD-fkbM, for removing both C9 hydroxylase and 31-O-methyltransferase, was transferred to Escherichia coli ET12567/pUZ8002 and then introduced into Streptomyces kanamyceticus by conjugation to delete the target gene by homologous recombination. Strains in which a single crossover occurred between the deletion plasmid and the Streptomyces kanamyceticus chromosome were selected by culturing an apramycin-resistant transconjugant in the presence of apramycin at 37° C. (non-growth tolerance temperature for pSG5-based replicon). Afterwards, the obtained colonies were propagated three times without selection at 28° C. to allow a second crossover.

Two achieved double crossover mutations, that is, ΔfkbD-fkbM, were selected as apramycin-sensitive expression traits, which were then confirmed by PCR and optionally by Southern block analysis.

A plasmid for modifying the C21 side chain, pΔtcsD, was introduced into the constructed Streptomyces kanamyceticus fkbD-fkbM lacking the fkbD-fkbM gene, and the tcsD gene was deleted using the same method as the fkbD-fkbM gene deletion method. ΔfkbD-fkbM,tcsD was selected as an apramycin-sensitive expression trait and then confirmed by PCR and optionally by Southern block analysis. By introducing pΔfkbL, which is a plasmid for forming a C1 prolyl ring, into the additionally constructed Streptomyces kanamyceticus ΔfkbD-fkbM,tcsD, in which the fkbD-fkbM and tcsD genes were deleted, the fkbL gene was deleted using the same method as the fkbD-fkbM and tcsD gene deletion method. ΔfkbD-fkbM,tcsD,fkbL was selected as an apramycin-sensitive expression trait and then confirmed by PCR.

The constructed fkbD-fkbM, tcsD, and fkbL gene deletion strain, Streptomyces kanamyceticus ΔfkbD-fkbM,tcsD,fkbL was deposited at the Korean Collection for Type Cultures (KCTC) on Apr. 14, 2020 (Accession No. KCTC14170BP).

9-Deoxo-31-O-demethyl-36,37-dihydro-prolylFK506 was prepared through the culture of the constructed production strain Streptomyces kanamyceticus fkbD-fkbM,tcsD,fkbL (Accession No. KCTC14170BP). It is specifically described as follows. In a 250 mL baffled flask, 50 mL of R2YE medium (sucrose 103 g/L, glucose 10 g/L, potassium sulfate 0.25 g/L, magnesium chloride hexahydrate 10.12 g/L, casamino acid 0.1 g/L, yeast extract (10%) 50 mL/L, TES buffer (5.73%, pH 7.2) 100 mL/L, potassium phosphate (0.5%) 10 mL/L, calcium chloride dihydrate (3.68%) 80 mL/L, L-proline (20%) 15 mL/L, trace element solution 2 mL/L, sodium hydroxide (1 N) 5 mL/L) was added, and the production strain was inoculated thereto, and pre-culture was carried out for two days in a rotary shaking incubator at 28° C. and 180 rpm. Next, 10 mL of the culture medium, which was pre-cultured for two days, was inoculated into a 3 L Erlenmeyer flask to which 1 L of R2YE medium was added. After inoculation, culture was performed for 6 days at 28° C. and 180 rpm. After culturing for 6 days, 9-deoxo-31-O-demethyl-36,37-dihydro-prolylFK506, which was produced through the primary recovery process, was extracted.

The primary recovery process was carried out as follows. First, the same amount of methanol was added to the culture medium and mixed for 30 minutes and centrifuged to remove cells, and the extract from which the cells were removed was concentrated using a rotary evaporator. Then, the concentrated extract was dissolved in water, ethyl acetate was added in a double volume, mixed well and then left to stand until the layers were separated. After the layers were separated, the organic solvent layer of the upper layer was recovered and concentrated using a rotary evaporator, and the weight after concentration was measured. The extract obtained by performing the primary recovery process was passed through a column filled with silica gel. In this case, the amount of silica gel was 15 times the weight of the extract in the primary recovery process, and the mobile phase was used at 5 ratios of n-hexene and ethyl acetate (fraction 1. 1:1, fraction 2. 1:2, fraction 3. 1:3, fraction 4. 0:1, fraction 5. methanol). In fraction 3, 9-deoxo-31-O-demethyl-36,37-dihydro-prolylFK506 was identified. Fraction 3 thus obtained was concentrated using a rotary evaporator and finally purified using HPLC.

It was freeze-dried to obtain 9-deoxo-31-O-demethyl-36,37-dihydro-prolylFK506, which is a substance represented by [Chemical Formula 2], in powder form.

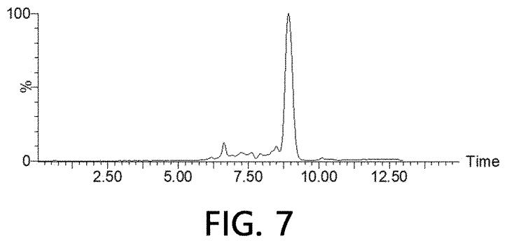

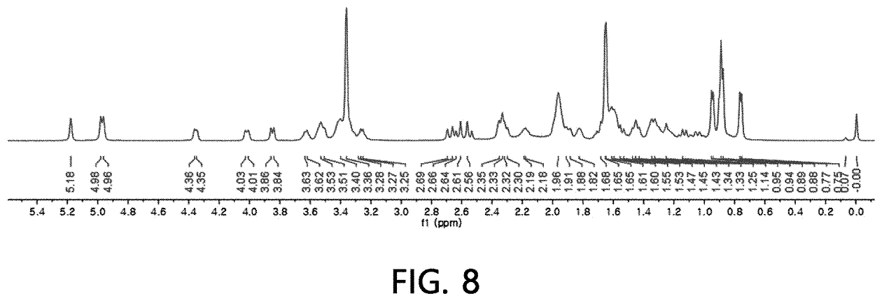

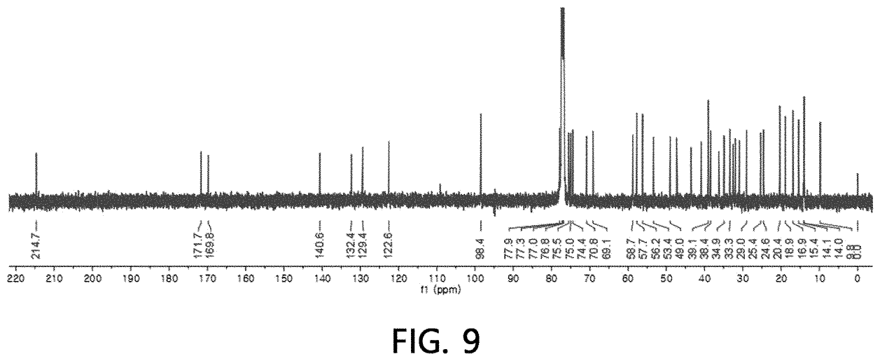

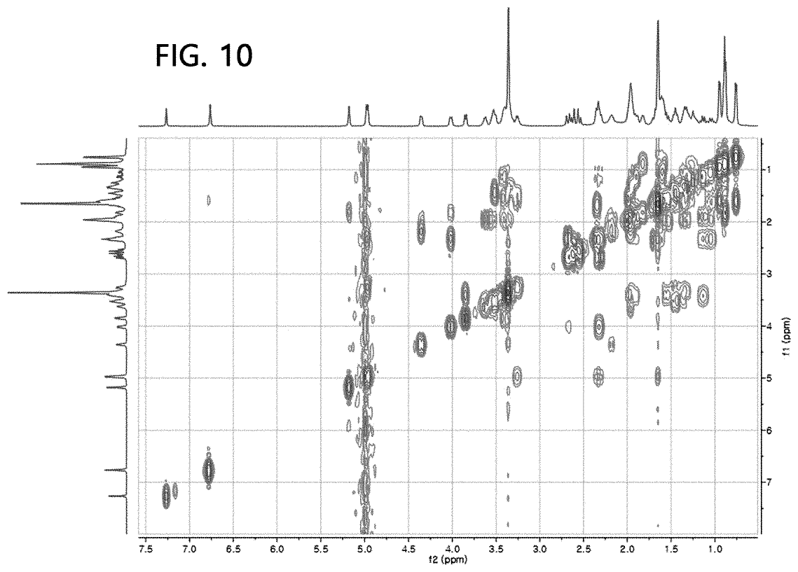

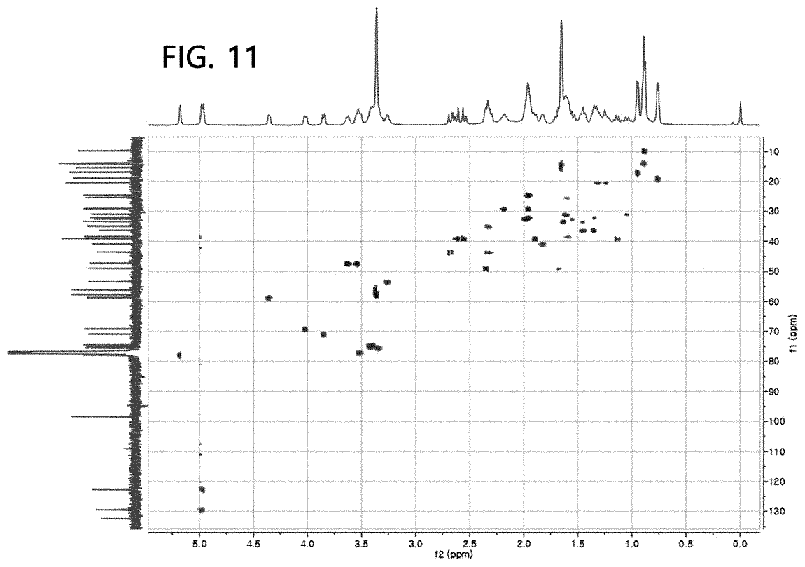

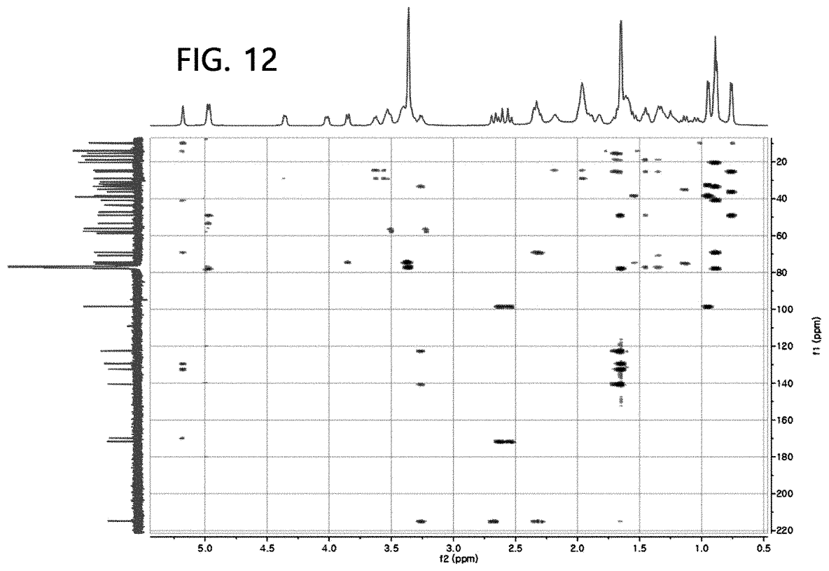

The confirmation of the prepared 9-deoxo-31-O-demethyl-36,37-dihydro-prolylFK506 was carried out as follows. Specifically, high performance liquid chromatography analysis, mass spectrometry and nuclear magnetic resonance analysis were performed. The analysis results for 9-deoxo-31-O-demethyl-36,37-dihydro-prolylFK506 are summarized in Table 4 and FIGS. 7 to 12, and from these results, it was confirmed that 9-deoxo-31-O-demethyl-36,37-dihydro-prolylFK506 was produced from the production strain Streptomyces kanamyceticus ΔfkbD-fkbM,tcsD,fkbL.

The analysis results for 9-deoxo-31-O-demethyl-36,37-dihydro-prolylFK506 (molecular formula: C₄₂H₇₃NO₁₁, molecular weight: 763.49) are shown in Table 4 below.

TABLE 4

| | Analysis results | | |
|---|---|---|---|
| | (ESI-HR-MS) Calcd. for C₄₂H₆₉NNaO₁₁⁺: | | |
| Analysis method | 786.4763, found: m/z 786.4767 | | |
| Mass spectrometry | No. | carbon (ppm) | proton (ppm) |
| Nuclear magnetic | 1 | 169.8 | |
| resonance analysis | 2 | 58.7 | 4.36 (1H, brd, J = 5.0 Hz) |
| | 3 | 29.0 | 1.97 (1H, m), 2.19 (1H, m) |
| | 4 | 24.8 | 1.97 (1H, m), 1.98 (1H, m) |
| | 5 | 47.3 | 3.52 (1H, m), 3.63 (1H, m) |
| | 6 | | |
| | 7 | | |
| | 8 | 171.7 | |
| | 9 | 39.1 | 2.54 (1H, d, J = 15.0 Hz), |
| | | | 2.63 (1H, d, J = 15.0 Hz) |
| | 10 | 98.4 | |
| | 11 | 38.4 | 1.59 (1H, m) |
| | 12 | 32.5 | 1.56 (1H, m), 1.98 (1H, m) |
| | 13 | 74.4 | 3.40 (1H, m) |
| | 14 | 70.8 | 3.85 (1H, brd, J = 10.0 Hz) |
| | 15 | 77.3 | 3.51 (1H, m) |
| | 16 | 36.3 | 1.34 (1H, m), 1.45 (1H, m) |
| | 17 | 25.4 | 1.60 (1H, m) |
| | 18 | 49.0 | 1.67 (1H, m), 2.35 (1H, m) |
| | 19 | 140.8 | |
| | 20 | 122.6 | 4.98 (1H, d, J = 5.0 Hz) |
| | 21 | 53.4 | 3.26 (1H, m) |
| | 22 | 216.3 | |
| | 23 | 43.5 | 2.31 (1H, brd J = 15.0 Hz), |
| | | | 2.68 (1H, brd J = 15.0 Hz) |
| | 24 | 69.1 | 4.02 (1H, m) |
| | 25 | 40.9 | 1.82 (1H, m) |
| | 26 | 77.9 | 5.18 (1H, brs) |
| | 27 | 132.4 | |
| | 28 | 129.4 | 4.97 (1H, d, J = 5.0 Hz) |
| | 29 | 34.9 | 2.32 (1H, m) |
| | 30 | 39.1 | 1.12 (1H, m), 1.90 (1H, m) |
| | 31 | 75.0 | 3.41 (1H, m) |
| | 32 | 75.5 | 3.34 (1H, m) |
| | 33 | 32.0 | 1.33 (1H, m), 1.95 (1H, m) |
| | 34 | 30.9 | 1.04 (1H, m), 1.61 (1H, m) |
| | 35 | 33.3 | 1.45 (1H, m), 1.63 (1H, m) |
| | 36 | 20.4 | 1.22 (2H, m) |
| | 37 | 14.0 | 0.88 (3H, t, J = 7.5 Hz) |
| | 38 | 16.9 | 0.95 (3H, d, J = 6.5 Hz) |
| | 39 | 18.9 | 0.76 (3H, d, J = 6.5 Hz) |
| | 40 | 15.4 | 1.65 (3H, s) |
| | 41 | 9.8 | 0.89 (3H, d, J = 6.5 Hz) |
| | 42 | 14.1 | 1.65 (3H, s) |
| | 43 | 57.7 | 3.36 (3H, s) |
| | 44 | 58.7 | 3.36 (3H, s) |

From ¹H and ¹³C-NMR, one ketone carbon (δ_{C} 216.3), two carbonyl carbons (δ_{C} 171.7, 169.8) and two olefine skeletons (δ_{C} 140.8, 122.6; δ_{C} 132.4, 129.4) were identified as characteristic functional groups, and dioxygenated quaternary carbon (δ_{C} 98.4), seven oxygenated methine carbons (δ_{C} 77.9, 77.3, 75.5, 75.0, 74.4, 70.8, 69.1) and two methoxy carbons (δ_{C} 58.7, 57.7) were observed, and six methyl carbons (δ_{C} 18.9, 16.9, 15.4, 14.1, 14.0, 9.8) were observed. In addition, all of 42 carbons were observed in the FK506 derivative. In order to determine the exact structure, 2D-NMR was confirmed. As a result of determining the connection of protons from gCOSY, it was confirmed from the coupling between H-2 to H-4 that this compound had a prolyl skeleton. From the gHMBC data, it was confirmed that this compound was a backbone reduced to CH₂ instead of ketone at C-9 from the correlation of H-9 (δ_{H} 2.54, 2.63) with C-8 (δ_{C} 171.7) and C-10 (δ_{C} 98.4). Together with this, it was confirmed that two methoxy functional groups were bonded to C-13 and C-15 such that it was a structure in which methoxy was not present at C-31.

In summary, it was confirmed that this compound was 9-deoxo-31-O-demethyl-36,37-dihydro-prolylFK506.

### EXAMPLE 3

### Preparation of 9-deoxo-prolylFK520

The in-frame deletion method by double cross-over homologous recombination according to the method described in Ban, Y. H. et al. (J. Nat. Prod. 2013, 76, 1091-1098) was used for Streptomyces kanamyceticus, which is a strain producing FK506, to cause the inactivation of fkbD, tcsD and fkbL genes to construct Streptomyces kanamyceticus ΔfkbD,tcsD,fkbL (Accession No. KCTC14171BP), which is a production strain of 9-deoxo-prolylFK520.

Specifically describing, in order to construct a deletion mutant of the fkbD, tcsD and fkbL genes in the Streptomyces kanamyceticus strain producing FK506, each gene was cloned into the pKC1139 vector and transferred to Escherichia coli ET12567/pUZ8002, and then, it was transformed into the FK506 producing strain Streptomyces kanamyceticus through conjugation.

The strain production method can be more specifically described as the construction of in-frame gene deletion plasmids and the production of gene deletion strains.

For the construction of in-frame gene deletion plasmids, E. coli-Streptomyces shuttle vector pKC1139 was used for in-frame gene deletion. Plasmid construction was performed by PCR amplification of the left- and right-flanking fragments of the target gene for deletion from Fosmid DNA derived from Streptomyces kanamyceticus. For deletion of the fkbD gene, a primer pair FkbDLF/FkbDLR for the left-adjacent fragment and a primer pair FkbDRF/FkbDRR for the right-adjacent fragment were designed. For deletion of the tcsD gene, a primer pair TcsDLF/TcsDLR for the left-adjacent fragment and a primer pair TcsDRF/TcsDRR for the right-adjacent fragment were designed. For deletion of the fkbL gene, a primer pair FkbLLF/FkbLLR for the left-adjacent fragment and a primer pair FkbLRF/FkbLRR for the right-adjacent fragment were designed. All PCR fragments were isolated, digested with HindIII-XbaI or XbaI-EcoRI and then cloned into pKC1139 vector. Information on the strains, plasmids and primers used in this example is presented in Tables 1 and 2.

The plasmids used to construct the gene deletion strain are summarized in Table 1. The plasmid, pΔfkbD, for removing C9 hydroxylase was transferred to Escherichia coli ET12567/pUZ8002 and then introduced into Streptomyces kanamyceticus by conjugation to delete the target gene by homologous recombination. Strains in which a single crossover occurred between the deletion plasmid and the Streptomyces kanamyceticus chromosome were selected by culturing an apramycin-resistant transconjugant in the presence of apramycin at 37° C. (non-growth tolerance temperature for pSG5-based replicon). Afterwards, the obtained colonies were propagated three times without selection at 28° C. to allow a second crossover. Two achieved double crossover mutations, that is, ΔfkbD, were selected as apramycin-sensitive expression traits, which were then confirmed by PCR and optionally by Southern block analysis.

A plasmid for modifying the C21 side chain, pΔtcsD, was introduced into the constructed Streptomyces kanamyceticus fkbD lacking the fkbD gene, and the tcsD gene was deleted using the same method as the fkbD gene deletion method. ΔfkbD,tcsD was selected as an apramycin-sensitive expression trait and then confirmed by PCR and optionally by Southern block analysis. By introducing pΔfkbL, which is a plasmid for forming a C1 prolyl ring, into the additionally constructed Streptomyces kanamyceticus ΔfkbD,tcsD, in which the fkbD and tcsD genes were deleted, the fkbL gene was deleted using the same method as the fkbD and tcsD gene deletion method. ΔfkbD,tcsD,fkbL were selected as apramycin-sensitive expression traits and then confirmed by PCR.

The constructed fkbD, tcsD, and fkbL gene deletion strain, Streptomyces kanamyceticus ΔfkbD,tcsD,fkbL was deposited at the Korean Collection for Type Cultures (KCTC) on Apr. 14, 2020 (Accession No. KCTC14171BP).

9-Deoxo-prolylFK520 was prepared through the culture of the constructed production strain Streptomyces kanamyceticus ΔfkbD,tcsD,fkbL (Accession No. KCTC14171BP). It is specifically described as follows. In a 250 mL baffled flask, 50 mL of R2YE medium (sucrose 103 g/L, glucose 10 g/L, potassium sulfate 0.25 g/L, magnesium chloride hexahydrate 10.12 g/L, casamino acid 0.1 g/L, yeast extract (10%) 50 mL/L, TES buffer (5.73%, pH 7.2) 100 mL/L, potassium phosphate (0.5%) 10 mL/L, calcium chloride dihydrate (3.68%) 80 mL/L, L-proline (20%) 15 mL/L, trace element solution 2 mL/L, sodium hydroxide (1 N) 5 mL/L) was added, and the production strain was inoculated thereto, and pre-culture was carried out for two days in a rotary shaking incubator at 28° C. and 180 rpm. Next, 10 mL of the culture medium, which was pre-cultured for two days, was inoculated into a 3 L Erlenmeyer flask to which 1 L of R2YE medium was added. After inoculation, culture was performed for 6 days at 28° C. and 180 rpm. After culturing for 6 days, 9-deoxo-prolylFK520, which was produced through the primary recovery process, was extracted.

The primary recovery process was carried out as follows. First, the same amount of methanol was added to the culture medium and mixed for 30 minutes and centrifuged to remove cells, and the extract from which the cells were removed was concentrated using a rotary evaporator. Then, the concentrated extract was dissolved in water, ethyl acetate was added in a double volume, mixed well and then left to stand until the layers were separated. After the layers were separated, the organic solvent layer of the upper layer was recovered and concentrated using a rotary evaporator, and the weight after concentration was measured. The extract obtained by performing the primary recovery process was passed through a column filled with silica gel. In this case, the amount of silica gel was 15 times the weight of the extract in the primary recovery process, and the mobile phase was used at 5 ratios of n-hexene and ethyl acetate (fraction 1. 1:1, fraction 2. 1:2, fraction 3. 1:3, fraction 4. 0:1, fraction 5. methanol). In fraction 3, 9-deoxo-prolylFK520 was identified. Fraction 3 thus obtained was concentrated using a rotary evaporator and finally purified using HPLC.

It was freeze-dried to obtain 9-deoxo-prolylFK520, which is a substance represented by [Chemical Formula 3], in powder form.

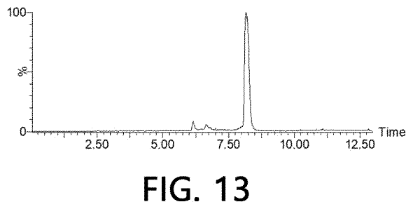

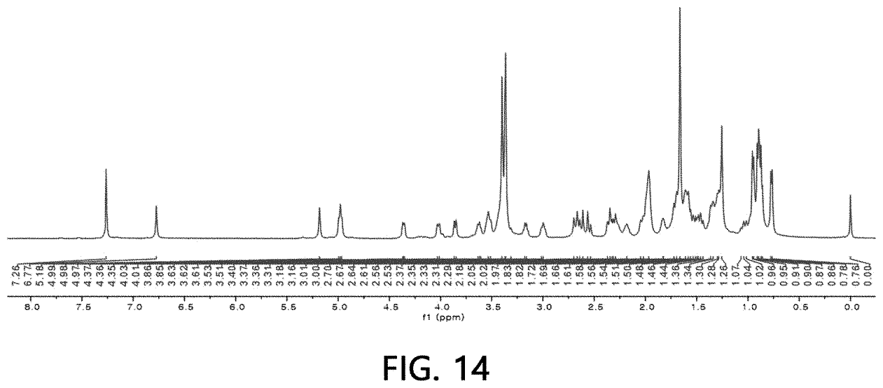

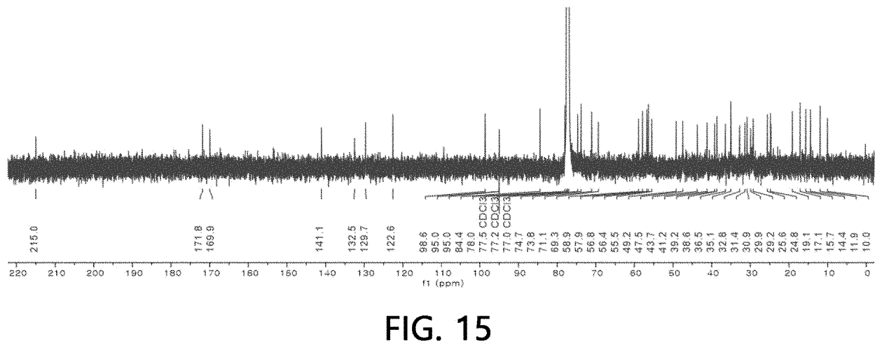

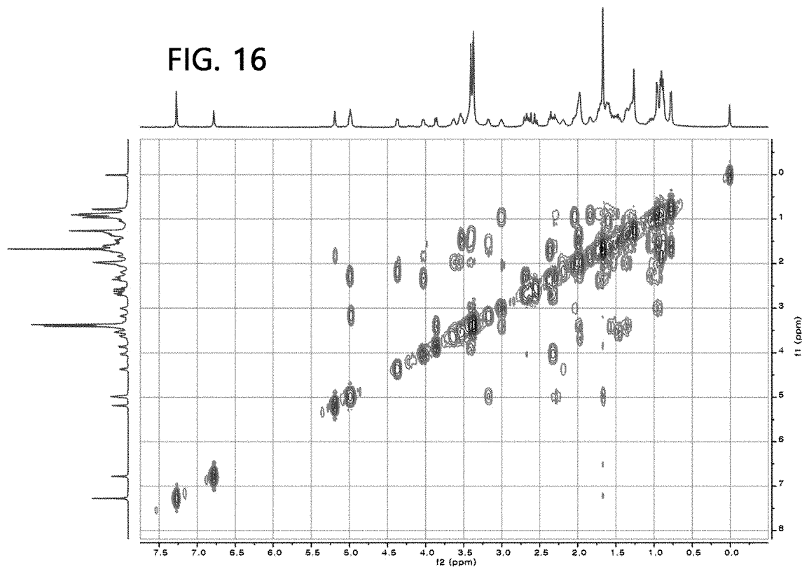

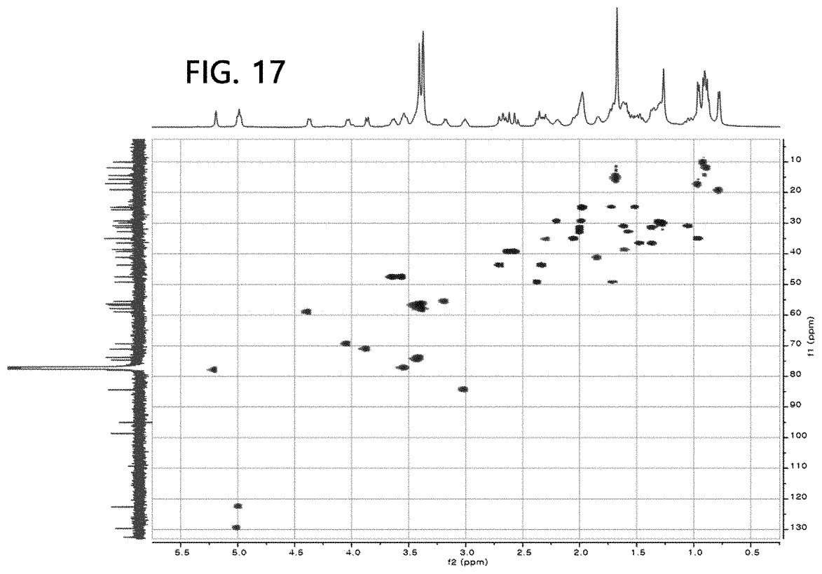

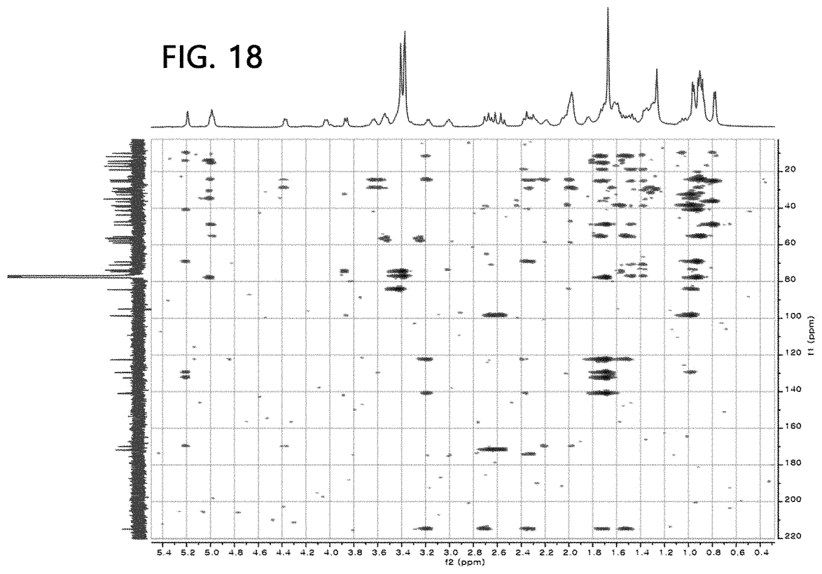

The confirmation of the prepared 9-deoxo-prolylFK520 was carried out as follows. Specifically, high performance liquid chromatography analysis, mass spectrometry and nuclear magnetic resonance analysis were performed. The analysis results for 9-deoxo-prolylFK520 are summarized in Table 5 and FIGS. 13 to 18, and from these results, it was confirmed that 9-deoxo-prolylFK520 was produced from the production strain Streptomyces kanamyceticus ΔfkbD,tcsD,fkbL.

The analysis results for 9-deoxo-prolylFK520 (molecular formula: C₄₂H₆₉NO₁₁, molecular weight: 763.49) are shown in Table 5 below.

TABLE 5

| | Analysis results | | |
|---|---|---|---|
| | (ESI-HR-MS) Calcd. for C₄₂H₆₉NNaO₁₁⁺: | | |
| Analysis method | 786.4763, found: m/z 786.4768 | | |
| Mass spectrometry | No. | carbon (ppm) | proton (ppm) |
| Nuclear magnetic | 1 | 169.9 | |
| resonance analysis | 2 | 58.9 | 4.37 (1H, brd, J = 5.0 Hz) |
| | 3 | 29.2 | 1.98 (1H, m), 2.20 (1H, m) |
| | 4 | 25.8 | 1.97 (1H, m), 1.98 (1H, m) |
| | 5 | 47.5 | 3.56 (1H, m), 3.65 (1H, m) |
| | 6 | | |
| | 7 | | |
| | 8 | 171.8 | |
| | 9 | 39.2 | 2.57 (1H, d, J = 15.0 Hz), |
| | | | 2.62 (1H, d, J = 15.0 Hz) |
| | 10 | 98.6 | |
| | 11 | 38.6 | 1.59 (1H, m) |
| | 12 | 32.8 | 1.56 (1H, m), 1.99 (1H, m) |
| | 13 | 74.4 | 3.40 (1H, m) |
| | 14 | 71.1 | 3.85 (1H, brd, J = 10.0 Hz) |
| | 15 | 77.4 | 3.54 (1H, m) |
| | 16 | 36.3 | 1.34 (1H, m), 1.45 (1H, m) |
| | 17 | 25.4 | 1.61 (1H, m) |
| | 18 | 49.2 | 1.67 (1H, m), 2.36 (1H, m) |
| | 19 | 141.1 | |
| | 20 | 122.6 | 4.99 (1H, d, J = 5.0 Hz) |
| | 21 | 55.5 | 3.18 (1H, m) |
| | 22 | 215.0 | |
| | 23 | 43.7 | 2.33 (1H, brd J = 15.0 Hz), |
| | | | 2.68 (1H, brd J = 15.0 Hz) |
| | 24 | 69.4 | 4.04 (1H, m) |
| | 25 | 41.2 | 1.83 (1H, m) |
| | 26 | 78.0 | 5.19 (1H, brs) |
| | 27 | 132.5 | |
| | 28 | 129.7 | 5.02 (1H, d, J = 5.0 Hz) |
| | 29 | 35.1 | 2.28 (1H, m) |
| | 30 | 35.0 | 0.95 (1H, m), 2.05 (1H, m) |
| | 31 | 84.2 | 3.01 (1H, m) |
| | 32 | 73.8 | 3.42 (1H, m) |
| | 33 | 31.4 | 1.36 (1H, m), 1.96 (1H, m) |
| | 34 | 30.9 | 1.03 (1H, m), 1.60 (1H, m) |
| | 35 | 24.8 | 1.51 (1H, m), 1.71 (1H, m) |
| | 36 | 11.9 | 0.88 (3H, t, J = 7.5 Hz) |
| | 37 | 17.1 | 0.96 (3H, d, J = 6.5 Hz) |
| | 38 | 19.1 | 0.78 (3H, d, J = 6.5 Hz) |
| | 39 | 15.7 | 1.67 (3H, s) |
| | 40 | 10.0 | 0.91 (3H, d, J = 6.5 Hz) |
| | 41 | 14.4 | 1.67 (3H, s) |
| | 42 | 56.4 | 3.36 (3H, s) |
| | 43 | 57.9 | 3.37 (3H, s) |
| | 44 | 56.8 | 3.40 (3H, s) |

From ¹H and ¹³C-NMR, one ketone carbon (δ_{C} 215.0), two carbonyl carbons (δ_{C} 171.8, 169.9) and two olefine skeletons (δ_{C} 141.1, 122.6; δ_{C} 132.5, 129.7) were identified as characteristic functional groups, and dioxygenated quaternary carbon (δ_{C} 98.6), seven oxygenated methine carbons (δ_{C} 84.2, 78.0, 77.4, 74.4, 73.8, 71.1, 69.4) and three methoxy carbons (δ_{C} 57.9, 57.9, 56.4) were observed, and six methyl carbons (δ_{C} 19.1, 17.1, 15.7, 14.4, 11.9, 10.0) were observed. In addition, all of 42 carbons were observed in the FK506 derivative. In order to determine the exact structure, 2D-NMR was confirmed. As a result of determining the connection of protons from gCOSY, it was confirmed from the coupling between H-2 to H-4 that this compound had a prolyl skeleton. From the gHMBC data, it was confirmed that this compound was a backbone reduced to CH₂ instead of ketone at C-9 from the correlation of H-9 (δ_{H} 2.57, 2.62) with C-8 (δ_{C} 171.8) and C-10 (δ_{C} 98.6). Together with this, it was confirmed that it was a structure in which three methoxy functional groups were present at C-13, C-15 and C-31. In addition, through gCOSY coupling correlation and gHMBC long range correlation, it was confirmed that it was a structure in which C-35 and C-36 were linked at C-21 with an ethyl group. In summary, it was confirmed that this compound was 9-deoxo-prolylFK520.

### EXAMPLE 4

### Preparation of 9-deoxo-31-O-demethyl-prolylFK520

The in-frame deletion method by double cross-over homologous recombination according to the method described in Ban, Y. H. et al. (J. Nat. Prod. 2013, 76, 1091-1098) was used for Streptomyces kanamyceticus, which is a strain producing FK506, to cause the inactivation of fkbD-fkbM, tcsD and fkbL genes to construct Streptomyces kanamyceticus ΔfkbD-fkbM,tcsD,fkbL (Accession No. KCTC14170BP), which is a production strain of 9-deoxo-31-O-demethyl-prolylFK520.

Specifically describing, in order to construct a deletion mutant of the fkbD-fkbD-fkbM, tcsD and fkbL genes in the Streptomyces kanamyceticus strain producing FK506, each gene was cloned into the pKC1139 vector and transferred to Escherichia coli ET12567/pUZ8002, and then, it was transformed into the FK506 producing strain Streptomyces kanamyceticus through conjugation.

The strain production method can be more specifically described as the construction of in-frame gene deletion plasmids and the production of gene deletion strains.

For the construction of in-frame gene deletion plasmids, E. coli-Streptomyces shuttle vector pKC1139 was used for in-frame gene deletion. Plasmid construction was performed by PCR amplification of the left- and right-flanking fragments of the target gene for deletion from Fosmid DNA derived from Streptomyces kanamyceticus. For deletion of the fkbD-fkbM gene, a primer pair FkbD-MLF/FkbD-MLR for the left-adjacent fragment and a primer pair FkbD-MRF/FkbD-MRR for the right-adjacent fragment were designed. For deletion of the tcsD gene, a primer pair TcsDLF/TcsDLR for the left-adjacent fragment and a primer pair TcsDRF/TcsDRR for the right-adjacent fragment were designed. For deletion of the fkbL gene, a primer pair FkbLLF/FkbLLR for the left-adjacent fragment and a primer pair FkbLRF/FkbLRR for the right-adjacent fragment were designed. All PCR fragments were isolated, digested with HindIII-XbaI or XbaI-EcoRI and then cloned into pKC1139 vector. Information on the strains, plasmids and primers used in this example is presented in Tables 1 and 2.

The plasmids used to construct the gene deletion strain are summarized in Table 1. The plasmid, pΔfkbD-fkbM, for removing both C9 hydroxylase and 31-O-methyltransferase, was transferred to Escherichia coli ET12567/pUZ8002 and then introduced into Streptomyces kanamyceticus by conjugation to delete the target gene by homologous recombination. Strains in which a single crossover occurred between the deletion plasmid and the Streptomyces kanamyceticus chromosome were selected by culturing an apramycin-resistant transconjugant in the presence of apramycin at 37° C. (non-growth tolerance temperature for pSG5-based replicon). Afterwards, the obtained colonies were propagated three times without selection at 28° C. to allow a second crossover. Two achieved double crossover mutations, that is, ΔfkbD-fkbM, were selected as apramycin-sensitive expression traits, which were then confirmed by PCR and optionally by Southern block analysis.

A plasmid for modifying the C21 side chain, pΔtcsD, was introduced into the constructed Streptomyces kanamyceticus ΔfkbD-fkbM lacking the fkbD-fkbM gene, and the tcsD gene was deleted using the same method as the fkbD gene deletion method. ΔfkbD-fkbM,tcsD was selected as an apramycin-sensitive expression trait and then confirmed by PCR and optionally by Southern block analysis. By introducing pΔfkbL, which is a plasmid for forming a C1 prolyl ring, into the additionally constructed Streptomyces kanamyceticus ΔfkbD-fkbM,tcsD, in which the fkbD-fkbM and tcsD genes were deleted, the fkbL gene was deleted using the same method as the fkbD-fkbM and tcsD gene deletion method. ΔfkbD-fkbM,tcsD,fkbL was selected as an apramycin-sensitive expression trait and then confirmed by PCR.

The constructed fkbD-fkbM,tcsD,fkbL gene deletion strain, Streptomyces kanamyceticus ΔfkbD-fkbM,tcsD,fkbL was deposited at the Korean Collection for Type Cultures (KCTC) on Apr. 14, 2020 (Accession No. KCTC14170BP).

9-Deoxo-31-O-demethyl-prolylFK520 was prepared through the culture of the constructed production strain Streptomyces kanamyceticus ΔfkbD-fkbM,tcsD,fkbL (Accession No. KCTC14170BP). It is specifically described as follows. In a 250 mL baffled flask, 50 mL of R2YE medium (sucrose 103 g/L, glucose 10 g/L, potassium sulfate 0.25 g/L, magnesium chloride hexahydrate 10.12 g/L, casamino acid 0.1 g/L, yeast extract (10%) 50 mL/L, TES buffer (5.73%, pH 7.2) 100 mL/L, potassium phosphate (0.5%) 10 mL/L, calcium chloride dihydrate (3.68%) 80 mL/L, L-proline (20%) 15 mL/L, trace element solution 2 mL/L, sodium hydroxide (1 N) 5 mL/L) was added, and the production strain was inoculated thereto, and pre-culture was carried out for two days in a rotary shaking incubator at 28° C. and 180 rpm. Next, 10 mL of the culture medium, which was pre-cultured for two days, was inoculated into a 3 L Erlenmeyer flask to which 1 L of R2YE medium was added. After inoculation, culture was performed for 6 days at 28° C. and 180 rpm. After culturing for 6 days, 9-deoxo-31-O-demethyl-prolylFK520, which was produced through the primary recovery process, was extracted.

The primary recovery process was carried out as follows. First, the same amount of methanol was added to the culture medium and mixed for 30 minutes and centrifuged to remove cells, and the extract from which the cells were removed was concentrated using a rotary evaporator. Then, the concentrated extract was dissolved in water, ethyl acetate was added in a double volume, mixed well and then left to stand until the layers were separated. After the layers were separated, the organic solvent layer of the upper layer was recovered and concentrated using a rotary evaporator, and the weight after concentration was measured. The extract obtained by performing the primary recovery process was passed through a column filled with silica gel. In this case, the amount of silica gel was 15 times the weight of the extract in the primary recovery process, and the mobile phase was used at 5 ratios of n-hexene and ethyl acetate (fraction 1. 1:1, fraction 2. 1:2, fraction 3. 1:3, fraction 4. 0:1, fraction 5. methanol). In fraction 3, 9-deoxo-31-O-demethyl-prolylFK520 was identified. Fraction 3 thus obtained was concentrated using a rotary evaporator and finally purified using HPLC.

It was freeze-dried to obtain 9-deoxo-31-O-demethyl-prolylFK520, which is a substance represented by [Chemical Formula 4], in powder form.

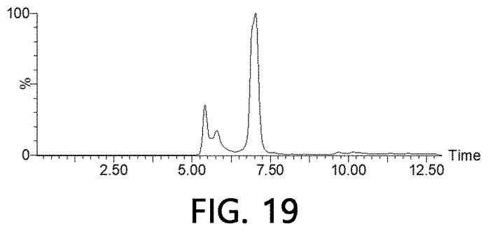

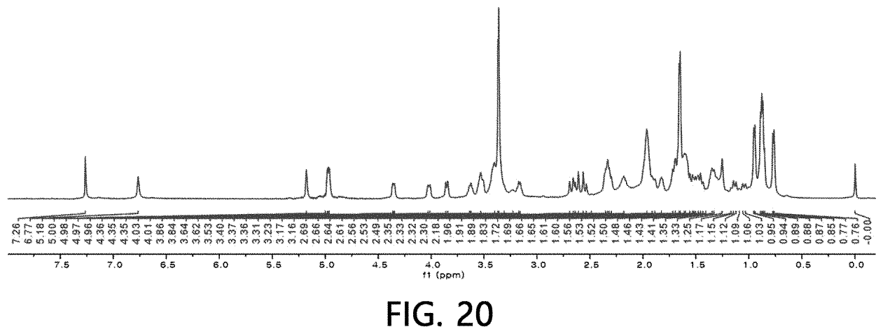

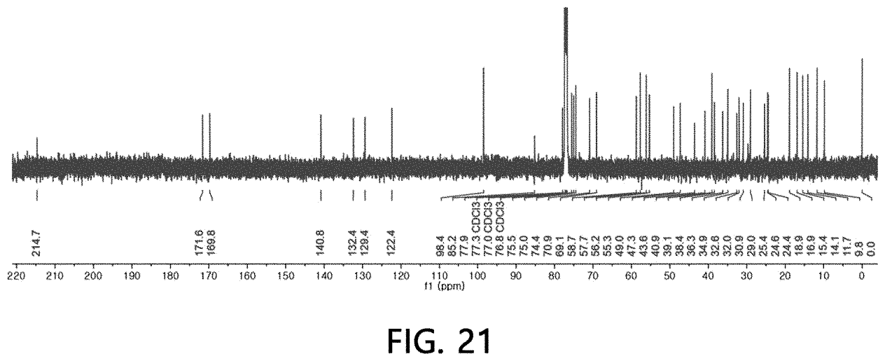

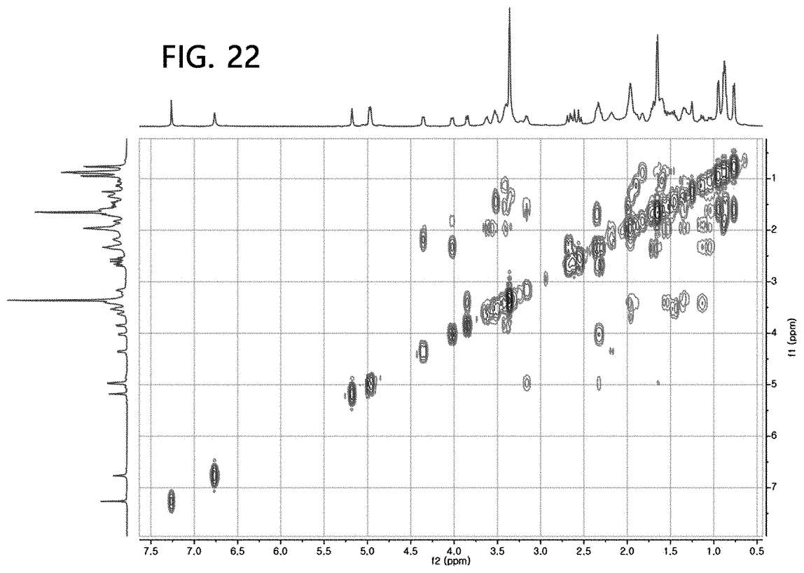

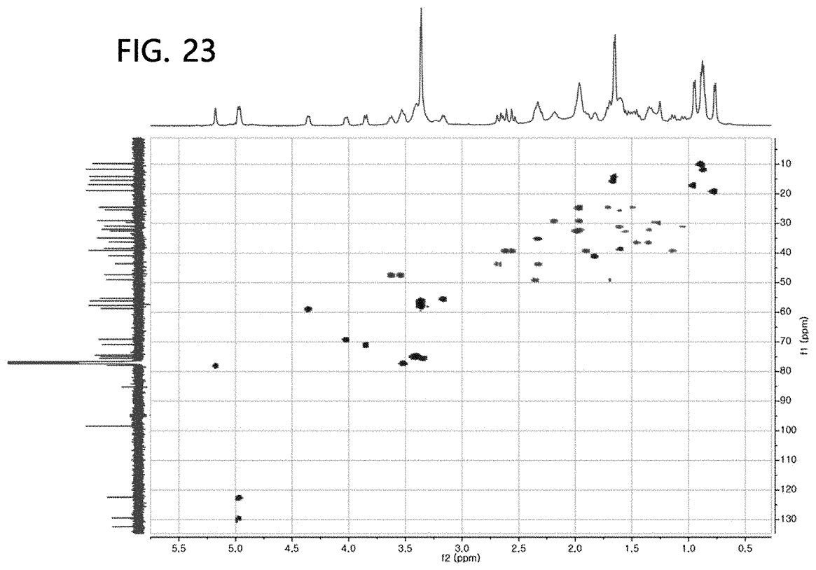

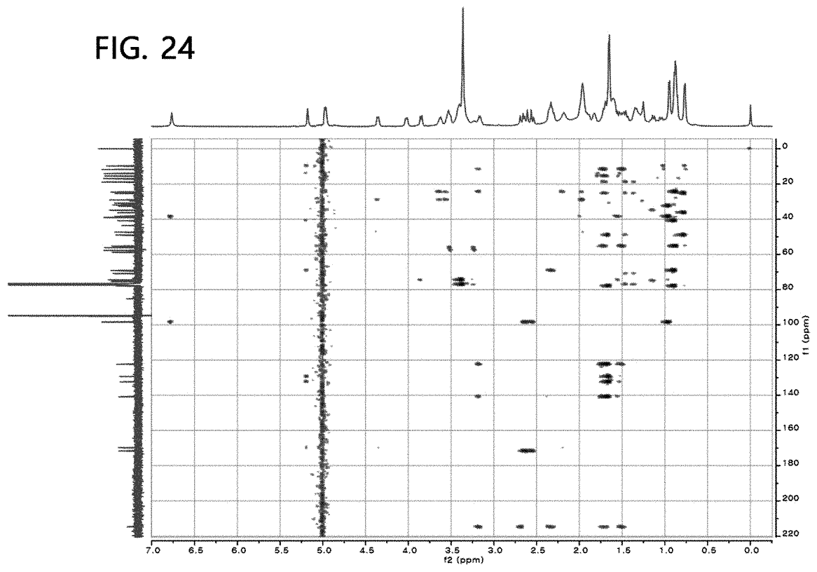

The confirmation of the prepared 9-deoxo-31-O-demethyl-prolylFK520 was carried out as follows. Specifically, high performance liquid chromatography analysis, mass spectrometry and nuclear magnetic resonance analysis were performed. The analysis results for 9-deoxo-31-O-demethyl-prolylFK520 are summarized in Table 6 and FIGS. 19 to 24, and from these results, it was confirmed that 9-deoxo-31-O-demethyl-prolylFK520 was produced from the production strain Streptomyces kanamyceticus ΔfkbD-fkbM,tcsD,fkbL.

The analysis results for 9-deoxo-31-O-demethyl-prolylFK520 (molecular formula: C₄₁H₆₇NO₁₁, molecular weight: 749.97) are shown in Table 6 below.

TABLE 6

| | Analysis results | | |
|---|---|---|---|
| | (ESI-HR-MS) Calcd. for C₄₁H₆₇NNaO₁₁⁺: | | |
| Analysis method | 772.4614, found: m/z 772.4619 | | |
| Mass spectrometry | No. | carbon (ppm) | proton (ppm) |
| Nuclear magnetic | 1 | 169.8 | |
| resonance analysis | 2 | 58.7 | 4.36 (1H, brd, J = 5.0 Hz) |
| | 3 | 29.0 | 1.96 (1H, m), 2.18 (1H, m) |
| | 4 | 25.4 | 1.97 (1H, m), 1.98 (1H, m) |
| | 5 | 47.3 | 3.54 (1H, m), 3.63 (1H, m) |
| | 6 | | |
| | 7 | | |
| | 8 | 171.6 | |
| | 9 | 39.1 | 2.56 (1H, d, J = 15.0 Hz), |
| | | | 2.62 (1H, d, J = 15.0 Hz) |
| | 10 | 98.4 | |
| | 11 | 38.4 | 1.59 (1H, m) |
| | 12 | 32.6 | 1.56 (1H, m), 1.98 (1H, m) |
| | 13 | 74.4 | 3.40 (1H, m) |
| | 14 | 70.9 | 3.85 (1H, brd, J = 10.0 Hz) |
| | 15 | 77.3 | 3.52 (1H, m) |
| | 16 | 36.3 | 1.34 (1H, m), 1.45 (1H, m) |
| | 17 | 25.4 | 1.60 (1H, m) |
| | 18 | 49.0 | 1.69 (1H, m), 2.35 (1H, m) |
| | 19 | 140.8 | |
| | 20 | 122.4 | 4.97 (1H, d, J = 5.0 Hz) |
| | 21 | 55.3 | 3.17 (1H, m) |
| | 22 | 214.7 | |
| | 23 | 43.8 | 2.32 (1H, brd J = 15.0 Hz), |
| | | | 2.66 (1H, brd J = 15.0 Hz) |
| | 24 | 69.1 | 4.02 (1H, m) |
| | 25 | 40.9 | 1.82 (1H, m) |
| | 26 | 77.9 | 5.18 (1H, brs) |
| | 27 | 132.4 | |
| | 28 | 129.4 | 4.97 (1H, d, J = 5.0 Hz) |
| | 29 | 34.9 | 2.32 (1H, m) |
| | 30 | 39.1 | 1.12 (1H, m), 1.90 (1H, m) |
| | 31 | 75.0 | 3.41 (1H, m) |
| | 32 | 75.5 | 3.34 (1H, m) |
| | 33 | 32.0 | 1.33 (1H, m), 1.95 (1H, m) |
| | 34 | 30.9 | 1.04 (1H, m), 1.61 (1H, m) |
| | 35 | 24.6 | 1.49 (1H, m), 1.72 (1H, m) |
| | 36 | 11.7 | 0.87 (3H, t, J = 7.5 Hz) |
| | 37 | 16.9 | 0.95 (3H, d, J = 6.5 Hz) |
| | 38 | 18.9 | 0.77 (3H, d, J = 6.5 Hz) |
| | 39 | 15.4 | 1.65 (3H, s) |
| | 40 | 9.8 | 0.89 (3H, d, J = 6.5 Hz) |
| | 41 | 14.1 | 1.65 (3H, s) |
| | 42 | 56.2 | 3.37 (3H, s) |
| | 43 | 57.7 | 3.37 (3H, s) |

From ¹H and ¹³C-NMR, one ketone carbon (δ_{C} 214.7), two carbonyl carbons (δ_{C} 171.6, 169.8) and two olefine skeletons (δ_{C} 140.8, 122.4; δ_{C} 132.4, 129.4) were identified as characteristic functional groups, and dioxygenated quaternary carbon (δ_{C} 98.4), seven oxygenated methine carbons (δ_{C} 77.9, 77.3, 75.5, 75.0, 74.4, 70.9, 69.1) and two methoxy carbons (δ_{C} 57.7, 56.2) were observed, and six methyl carbons (δ_{C} 18.9, 16.9, 15.4, 14.1, 11.7, 9.8) were observed. In addition, all of 41 carbons were observed in the FK506 derivative in which the carbon number was reduced. In order to determine the exact structure, 2D-NMR was confirmed. As a result of determining the connection of protons from gCOSY, it was confirmed from the coupling between H-2 to H-4 that this compound had a prolyl skeleton, and from the coupling correlation of H21-H35-36, it was confirmed to have the form of FK520. From the gHMBC data, it was confirmed that this compound was a backbone reduced to CH₂ instead of ketone at C-9 from the correlation of H-9 (δ_{H} 2.56, 2.62) with C-8 (δ_{C} 171.6) and C-10 (δ_{C} 98.4). Together with this, it was confirmed that two methoxy functional groups were bonded to C-13 and C-15 such that it was a structure in which methoxy was not present at C-31. In summary, it was confirmed that this compound was 9-deoxo-31-O-demethyl-prolylFK520.

### EXAMPLE 5

Investigation of Immunosuppressive Activity of Four Novel Compounds

The degree of decrease in immunosuppressive activity of the four novel compounds was investigated by using the conventional in vitro T-cell activity assay (J. Immunol. 143: 718-726, 1989). The division of CD4+ T cells is an indicator that an immune response is taking place, and when CD4+ T cells are stained with Cell Trace™ Violet (CTV) and the cells divide according to the immune response and the T cells proliferate, a phenomenon in which the CTV retention of each cell decreases appears, and thus, the degree of immunosuppressive activity was investigated using this as an indicator.

Single cells were isolated from the spleen of 6 to 8 week-old B6J mice, and CD4+ T cells were isolated using the MagniSort® Mouse CD4 T cell Enrichment Kit (eBioscience). CD4+ T cells were stained with Cell Trace™ Violet (CTV) Cell Proliferation Kit (Molecular Probes), and FK506 or the four novel compounds were added to a concentration of 0.01 ng/mL, 0.1 ng/mL, 1 ng/mL, 10 ng/mL, 100 ng/mL or 100 ng/mL, and then, it was cultured for 72 hours. Dynabeads® Mouse T-Activator CD3/CD28 (Gibco) was used for T cell activation. As a control group, non-activated T cells were used. After culture, CTV intensity was analyzed by flow cytometry.

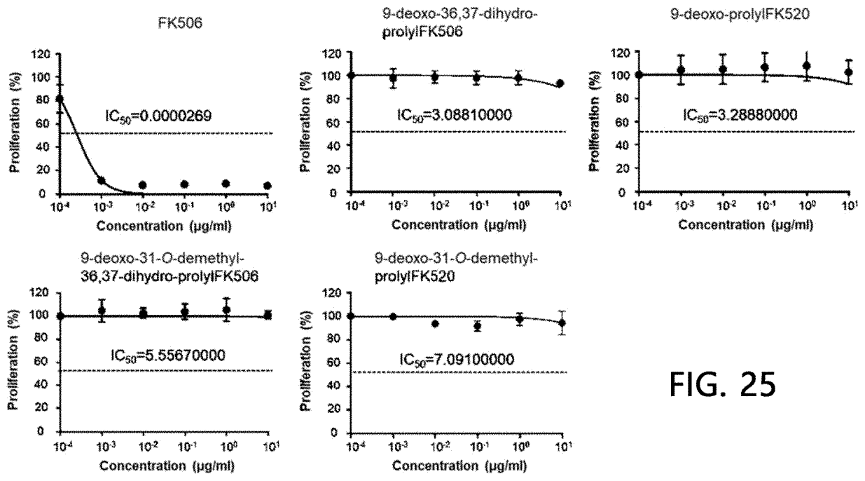

Table 7 and FIG. 25 below show the degree of T cell proliferation as measured by CTV intensity using a flow cytometer, and it shows the degree of immunosuppressive activity of FK506 and the four novel compounds. As shown in Table 7 and FIG. 25 below, all of the novel compounds presented in the present invention exhibited reduced immunosuppressive activity compared to FK506.

TABLE 7

| | Immunosuppression |
|---|---|
| Structural analogs | IC₅₀ (ng/mL) |
| FK506 | 0.027 |
| 9-deoxo-36,37-dihydro-prolylFK506 | 3088.1 |
| 9-deoxo-31-O-demethyl-36,37-dihydro- | 5556.7 |
| prolylFK506 | |
| 9-deoxo-prolylFK520 | 3288.8 |
| 9-deoxo-31-O-demethyl-prolylFK520 | 7091.0 |

From these results, it was confirmed that the immunosuppressive activity of the four novel compounds according to the present invention was significantly reduced compared to FK506, and the four new compounds showed an IC₅₀ (ng/mL) concentration of at least 1.14×10⁵ times or more. Accordingly, it was confirmed that the immunosuppressive activity was significantly reduced. From this, it was determined that a pharmaceutical composition for promoting hair growth, including at least one selected from the four novel compounds as an active ingredient, may be used without concern about side effects due to its immunosuppressive activity.

### EXAMPLE 6

Investigation of Hair Growth Activity Using Human Hair Follicles

The hair growth promoting effect in an ex vivo model system using human hair follicles was determined for the four novel compounds of the present invention. The test method is as follows.

In order to extract healthy-looking hair follicles from human scalp tissue, the tissue was first trimmed using a scalpel. From each trimmed hair follicle, the tissue surrounding the hair follicle was cut and removed with a scalpel, and each hair follicle was extracted cleanly. The extracted hair follicles were treated with FK506 (10 μM) or the four novel compounds (1, 10, 50 μM) (Table 8). The number of hair follicles in each group was 10, and the control group was treated with the same amount of 0.1% DMSO in the culture medium used.

TABLE 8

| Group | Sample |
|---|---|
| Control | 0.1% DMSO |
| Positive control | FK506 |
| Experimental Group 1 | 9-deoxo-36,37-dihydro-prolylFK506 |
| Experimental Group 2 | 9-deoxo-31-O-demethyl-36,37-dihydro- |
| | prolylFK506 |
| Experimental Group 3 | 9-deoxo-prolylFK520 |
| Experimental Group 4 | 9-deoxo-31-O-demethyl-prolylFK520 |

In an independent tube, the Williams'E culture medium and sample added with penicillin-streptomycin (100 U/mL) and the like were added and mixed well, and then, 250 μL of the culture medium/sample mixture was added to each well. The extracted hair follicles were placed into each well of a 48-well plate, and 10 samples were prepared for each group. Each well plate that was treated with each sample was placed in a 37° C. cell incubator and cultured. After 3 days of culture, the length of the hair follicles in each group was measured. The hair follicle length was compared by deriving the difference between the hair follicle length on day 0 and the length of the hair follicle on day 3 after culture in each group.

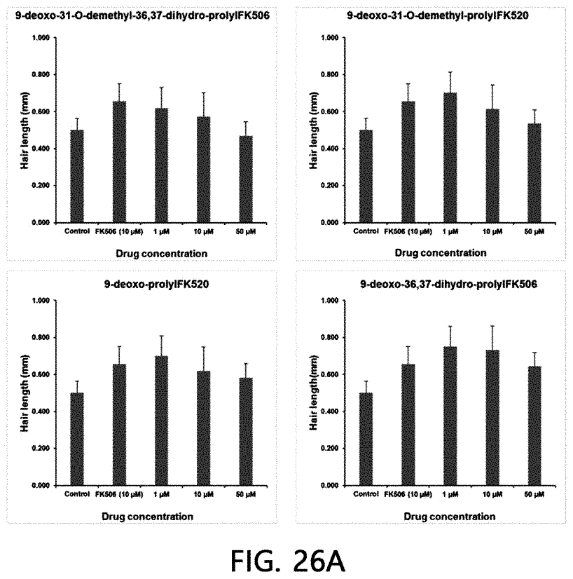

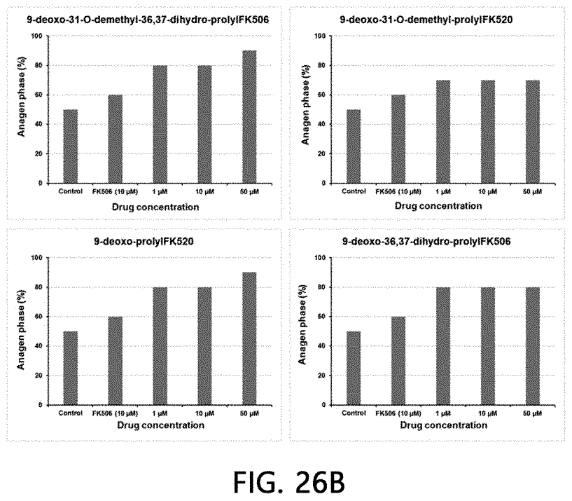

As confirmed in FIG. 26a, compared with FK506 (10 μM), the two tested compounds showed equivalent or superior hair growth promoting effect even at a lower concentration (1 μM). In addition, as a result of observing the degree of remaining hair follicles in the anagen phase before entering the catagen phase, during which the hair follicles fall out, all of the tested compounds showed an effect of prolonging the anagen phase, as confirmed in FIG. 26b. These results indicate that all four compounds of the present invention are effective in preventing hair loss.

From these results, it was confirmed that the four new compounds according to the present invention have improved hair growth activity compared to FK506, and the four new compounds have significantly reduced immunosuppressive activity because they showed an IC₅₀ (ng/mL) concentration of at least 1.14×10⁵ times or more. From this, it was determined that a pharmaceutical composition for preventing or treating hair loss, including at least one selected from the four novel compounds as an active ingredient, may be used without concern about side effects due to its hair growth activity.

From the foregoing, one of ordinary skill in the art to which the present disclosure pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the scope of the present invention is intended to cover not only the exemplary embodiments but also various alternatives, modifications, equivalents, and other embodiments that may be included within the spirit and scope of the present invention as defined by the appended claims.

### [Accession No.]

Name of Depositary Institution: Korea Research Institute of Bioscience and Biotechnology

Address of Depositary Institution: Korea Research Institute of Bioscience and Biotechnology (KRIBB), 181, Ipsin-gil, Jeongeup-si, Jeollabuk-do, 56212, Republic of Korea

Accession No.: KCTC14170BP

Deposit Date: Apr. 14, 2020

Name of Depositary Institution: Korea Research Institute of Bioscience and Biotechnology

Address of Depositary Institution: Korea Research Institute of Bioscience and Biotechnology (KRIBB), 181, Ipsin-gil, Jeongeup-si, Jeollabuk-do, 56212, Republic of Korea

Accession No.: KCTC14171BP

Deposit Date: Apr. 14, 2020

1. (canceled)

2. (canceled)

3. (canceled)

4. A method for ameliorating, preventing or treating hair loss, comprising administering a composition, which comprises any one compound selected from the group consisting of 9-deoxo-36,37-dihydro-prolylFK506 represented by [Chemical Formula 1] below, 9-deoxo-31-O-demethyl-36,37-dihydro-prolylFK506 represented by [Chemical Formula 2] below, 9-deoxo-prolylFK520 represented by [Chemical Formula 3] below and 9-deoxo-31-O-demethyl-prolylFK520 represented by [Chemical Formula 4] below, an isomer thereof or a pharmaceutically acceptable salt thereof as an active ingredient, to a subject:

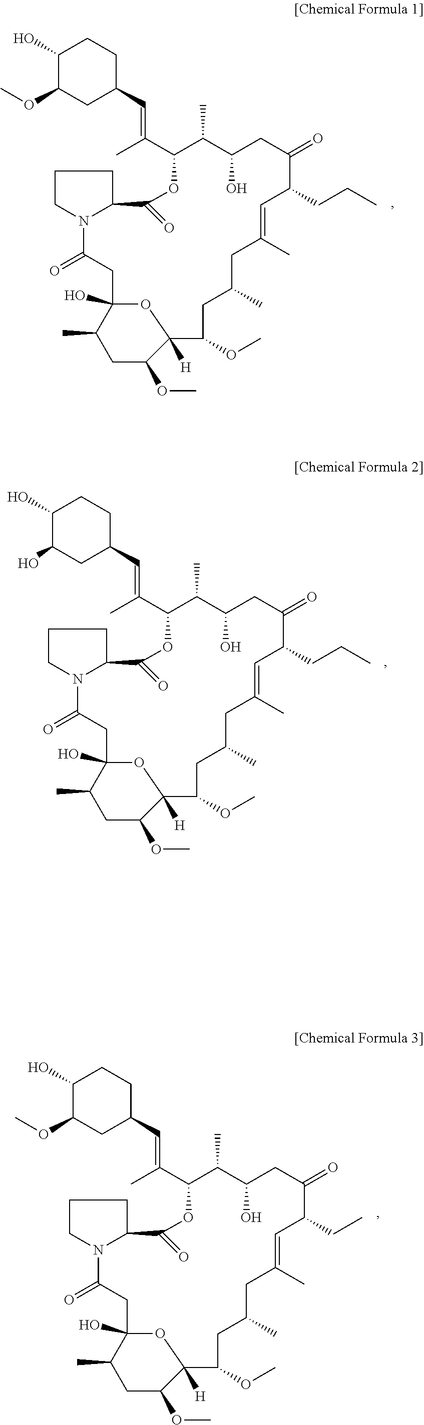

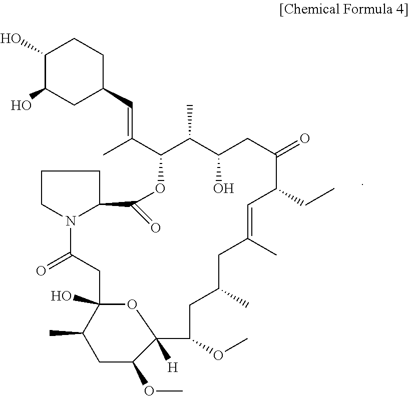 [SMILES: CC[C@@H]1/C=C(\C)C[C@H](C)C[C@H](OC)[C@H]2O[C@@](O)(CC(=O)N3CCC[C@H]3C(=O)O[C@H](/C(C)=C/[C@@H]3CC[C@@H](O)[C@H](O)C3)[C@H](C)[C@@H](O)CC1=O)[C@H](C)C[C@@H]2OC]

5. The method of claim 4, wherein the hair loss includes scarring hair loss or at least one non-scarring hair loss selected from the group consisting of infectious hair loss, traumatic hair loss, inflammatory hair loss, congenital hair loss, endocrine hair loss, neoplastic alopecia, malnutrition hair loss, drug-induced hair loss and hair loss due to structural abnormalities of hair, male-pattern hair loss, female-pattern hair loss and alopecia areata.

6. The method of claim 4, wherein the compound, isomer thereof or salt thereof exhibits an effect of prolonging the anagen stage in the hair cycle.

7. (canceled)

8. (canceled)

9. (canceled)

10. (canceled)

11. (canceled)

12. The method of claim 4, wherein the composition comprises a formulation of a medicament, a quasi-drug, health functional food or a cosmetic.

13. The method of claim 4, wherein the salt is a pharmaceutically acceptable salt, a sitologically acceptable salt or a cosmetically acceptable salt.

14. (canceled)

15. (canceled)

16. The method of claim 4, the quasi-drug comprises a formulation of a scalp tonic, a scalp lotion, a scalp cream, a scalp serum, a scalp essence, a scalp ampoule, a scalp treatment, a scalp conditioner, a scalp shampoo, a scalp pack, a hair tonic, a hair lotion, a hair cream, a hairspray, a hair mousse, a hair gel, a hair conditioner, a hair shampoo, a hair conditioner, a hair pack, a hair treatment, an eyebrow hair growth agent, an eyelash hair growth agent, an eyelash nutritional supplement, a pet shampoo or a pet rinse.

17. A method for promoting hair growth, comprising administering a composition, which comprises any one compound selected from the group consisting of 9-deoxo-36,37-dihydro-prolylFK506 represented by [Chemical Formula 1] below, 9-deoxo-31-O-demethyl-36,37-dihydro-prolylFK506 represented by [Chemical Formula 2] below, 9-deoxo-prolylFK520 represented by [Chemical Formula 3] below and 9-deoxo-31-O-demethyl-prolylFK520 represented by [Chemical Formula 4] below, an isomer thereof or a salt thereof as an active ingredient, to a subject:

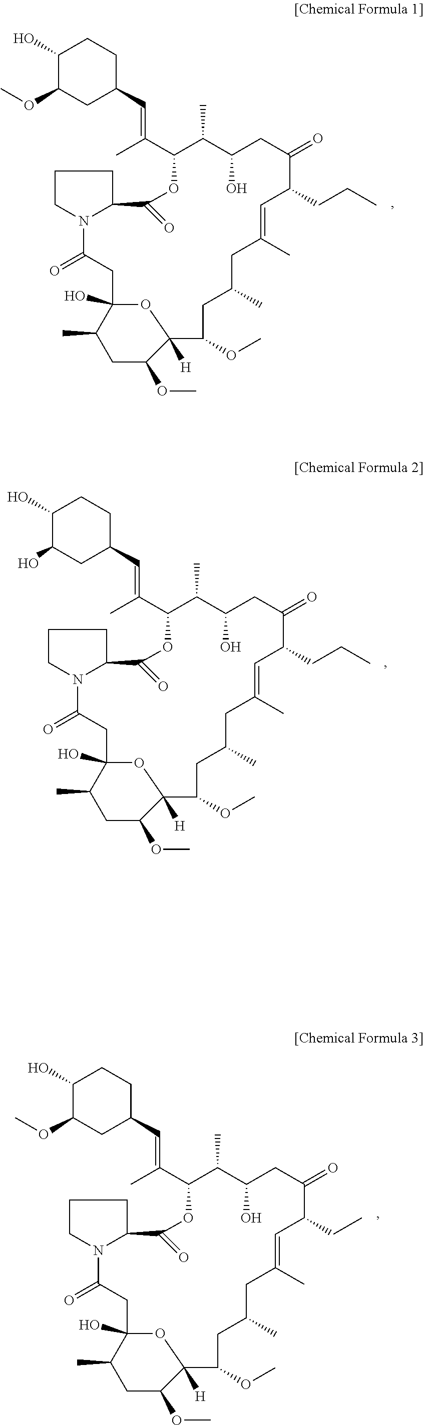

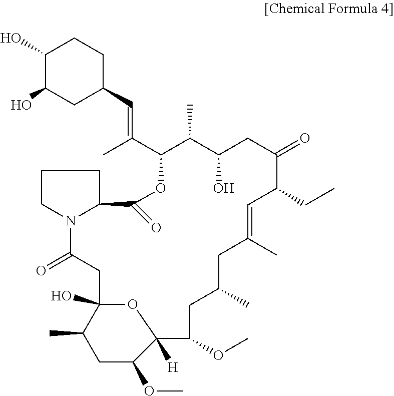 [SMILES: CC[C@@H]1/C=C(\C)C[C@H](C)C[C@H](OC)[C@H]2O[C@@](O)(CC(=O)N3CCC[C@H]3C(=O)O[C@H](/C(C)=C/[C@@H]3CC[C@@H](O)[C@H](O)C3)[C@H](C)[C@@H](O)CC1=O)[C@H](C)C[C@@H]2OC]

18. The method of claim 17, wherein the compound, isomer thereof or salt thereof exhibits an effect of prolonging the anagen stage in the hair cycle.

19. The method of claim 17, wherein the composition comprises a formulation of a medicament, a quasi-drug, health functional food or a cosmetic.

20. The method of claim 17, wherein the salt is a pharmaceutically acceptable salt, a sitologically acceptable salt or a cosmetically acceptable salt.

21. The method of claim 17, the quasi-drug comprises a formulation of a scalp tonic, a scalp lotion, a scalp cream, a scalp serum, a scalp essence, a scalp ampoule, a scalp treatment, a scalp conditioner, a scalp shampoo, a scalp pack, a hair tonic, a hair lotion, a hair cream, a hairspray, a hair mousse, a hair gel, a hair conditioner, a hair shampoo, a hair conditioner, a hair pack, a hair treatment, an eyebrow hair growth agent, an eyelash hair growth agent, an eyelash nutritional supplement, a pet shampoo or a pet rinse.
